(19) United States
(12) Patent Application Publication
(10) Pub. No.: US 2025/0127724 A1
(43) Pub. Date: Apr. 24, 2025

(54) SUSTAINED RELEASE DOSAGE FORMS FOR A JAK1 INHIBITOR

(71) Applicants: Incyte Corporation, Wilmington, DE (US); Incyte Holdings Corporation, Wilmington, DE (US)

(72) Inventors: Krishnaswamy Yeleswaram, Landenberg, PA (US); Bhavnish Parikh, Avondale, PA (US); Dilip P. Modi, Newark, DE (US); Trupti Sheth, Newark, DE (US)

(21) Appl. No.: 18/897,376

(22) Filed: Sep. 26, 2024

Related U.S. Application Data
(63) Continuation of application No. 17/333,224, filed on May 28, 2021, now Pat. No. 12,151,026.
Application No. 17/333,224 is a continuation of application No. 16/746,399, filed on Jan. 17, 2020, now Pat. No. 11,045,421.
Application No. 16/746,399 is a continuation of application No. 15/599,084, filed on May 18, 2017, now Pat. No. 10,561,616.
Application No. 15/599,084 is a continuation of application No. 14/453,129, filed on Aug. 6, 2014, now Pat. No. 9,655,854.
(60) Provisional application No. 61/913,066, filed on Dec. 6, 2013.
(60) Provisional application No. 61/863,325, filed on Aug. 7, 2013.

(51) Int. Cl. A61K 9/20; A61K 31/519

(52) U.S. Cl. CPC A61K 9/2054; A61K 9/2009; A61K 9/2013; A61K 9/2018; A61K 9/2031; A61K 9/2059; A61K 31/519

(57) ABSTRACT

This invention relates to sustained release dosage forms comprising {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof, and doses and methods related thereto.

This application is a continuation of U.S. patent application Ser. No. 17/333,224, filed on May 28, 2021, which is a continuation of U.S. patent application Ser. No. 16/746,399, filed on Jan. 17, 2020, now issued U.S. Pat. No. 11,045,421, which is a continuation of U.S. patent application Ser. No. 15/599,084, filed May 18, 2017, now issued U.S. Pat. No. 10,561,616, which is a continuation of U.S. patent application Ser. No. 14/453,129, filed Aug. 6, 2014, now issued U.S. Pat. No. 9,655,854, which claims the benefit of priority of U.S. Prov. Appl. No. 61/863,325, filed Aug. 7, 2013, and U.S. Prov. Appl. No. 61/913,066, filed Dec. 6, 2013, each of which is incorporated herein by reference in its entirety.

### TECHNICAL FIELD

This application relates to a sustained release dosage form comprising {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof, and doses and methods related thereto.

### BACKGROUND

Protein kinases (PKs) regulate diverse biological processes including cell growth, survival, differentiation, organ formation, morphogenesis, neovascularization, tissue repair, and regeneration, among others. Protein kinases also play specialized roles in a host of human diseases including cancer. Cytokines, low-molecular weight polypeptides or glycoproteins, regulate many pathways involved in the host inflammatory response to sepsis. Cytokines influence cell differentiation, proliferation and activation, and can modulate both pro-inflammatory and anti-inflammatory responses to allow the host to react appropriately to pathogens. Signaling of a wide range of cytokines involves the Janus kinase family (JAKs) of protein tyrosine kinases and Signal Transducers and Activators of Transcription (STATs). There are four known mammalian JAKs: JAK1 (Janus kinase-1), JAK2, JAK3 (also known as Janus kinase, leukocyte; JAKL; and L-JAK), and TYK2 (protein-tyrosine kinase 2).

Cytokine-stimulated immune and inflammatory responses contribute to pathogenesis of diseases: pathologies such as severe combined immunodeficiency (SCID) arise from suppression of the immune system, while a hyperactive or inappropriate immune/inflammatory response contributes to the pathology of autoimmune diseases (e.g., asthma, systemic lupus erythematosus, thyroiditis, myocarditis), and illnesses such as scleroderma and osteoarthritis (Ortmann, R. A., T. Cheng, et al. (2000) Arthritis Res 2(1): 16-32).

Deficiencies in expression of JAKs are associated with many disease states. For example, Jak1-/-mice are runted at birth, fail to nurse, and die perinatally (Rodig, S. J., M. A. Meraz, et al. (1998) Cell 93(3): 373-83). Jak2−/− mouse embryos are anemic and die around day 12.5 postcoitum due to the absence of definitive erythropoiesis.

The JAK/STAT pathway, and in particular all four JAKs, are believed to play a role in the pathogenesis of asthmatic response, chronic obstructive pulmonary disease, bronchitis, and other related inflammatory diseases of the lower respiratory tract. Multiple cytokines that signal through JAKs have been linked to inflammatory diseases/conditions of the upper respiratory tract, such as those affecting the nose and sinuses (e.g., rhinitis and sinusitis) whether classically allergic reactions or not. The JAK/STAT pathway has also been implicated in inflammatory diseases/conditions of the eye and chronic allergic responses.

Activation of JAK/STAT in cancers may occur by cytokine stimulation (e.g. IL-6 or GM-CSF) or by a reduction in the endogenous suppressors of JAK signaling such as SOCS (suppressor or cytokine signaling) or PIAS (protein inhibitor of activated STAT) (Boudny, V., and Kovarik, J., Neoplasm. 49:349-355, 2002). Activation of STAT signaling, as well as other pathways downstream of JAKs (e.g., Akt), has been correlated with poor prognosis in many cancer types (Bowman, T., et al. Oncogene 19:2474-2488, 2000). Elevated levels of circulating cytokines that signal through JAK/STAT play a causal role in cachexia and/or chronic fatigue. As such, JAK inhibition may be beneficial to cancer patients for reasons that extend beyond potential anti-tumor activity. JAK2 tyrosine kinase can be beneficial for patients with myeloproliferative disorders, e.g., polycythemia vera (PV), essential thrombocythemia (ET), myeloid metaplasia with myelofibrosis (MMM) (Levin, et al., Cancer Cell, vol. 7, 2005:387-397). Inhibition of the JAK2V617F kinase decreases proliferation of hematopoietic cells, suggesting JAK2 as a potential target for pharmacologic inhibition in patients with PV, ET, and MMM.

Inhibition of the JAKs may benefit patients suffering from skin immune disorders such as psoriasis, and skin sensitization. The maintenance of psoriasis is believed to depend on a number of inflammatory cytokines in addition to various chemokines and growth factors (JCI, 113:1664-1675), many of which signal through JAKs (Adv Pharmacol. 2000;47:113-74).

Due to the usefulness of compounds which inhibit JAK in targeting augmentation or suppression of the immune and inflammatory pathways (such as immunosuppressive agents for organ transplants), as well as the treatment of autoimmune diseases, diseases involving a hyperactive inflammatory response (e.g., eczema), allergies, cancer (e.g., prostate, leukemia, multiple myeloma), and some immune reactions (e.g., skin rash or contact dermatitis or diarrhea) caused by other therapeutics, there is a need for improved formulations for administering JAK kinases. The dosages forms described herein, as well as the doses and methods described supra are directed toward this need and other ends.

### SUMMARY

JAK inhibitors are described in U.S. Ser. No. 13/043,986 (US 2011/0224190), filed Mar. 9, 2011, which is incorporated herein by reference in its entirety, including {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, which is depicted below as Formula I.

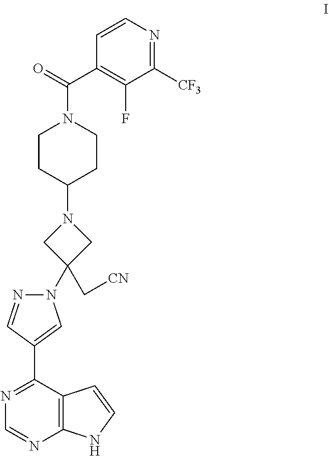 [SMILES: N#CCC1(n2cc(-c3ncnc4[nH]ccc34)cn2)CN(C2CCN(C(=O)c3ccnc(C(F)(F)F)c3F)CC2)C1]

The present application provides, inter alia, sustained-release dosage forms comprising about 25 mg to about 600 mg (e.g., 25 mg, 100 mg, 200 mg, 300 mg, or 600 mg) on a free base basis of {1-{1-[3-fluoro-2-(trifluoromethyl)isonicotinoyl]piperidin-4-y1}-3-[4-(7H-pyrrolo[2,3-d]pyrimidin-4-yl)-1H-pyrazol- 1-yl]azetidin-3-yl}acetonitrile, or a pharmaceutically acceptable salt thereof.

The present invention further provides one or more sustained release dosage forms each comprising {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof; wherein said one or more sustained release dosage forms together provide a once-daily oral dosage of about 400 mg to about 600 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof, to a patient.

The present invention also provides a dose, comprising one or more sustained release dosage forms each comprising {1-{1-[3-fluoro-2-(trifluoromethyl)isonicotinoyl]piperidin-4-yl}-3-[4-(7H-pyrrolo[2,3-d]pyrimidin-4-yl)-1H- pyrazol-1-yl]azetidin-3-yl}acetonitrile, or a pharmaceutically acceptable salt thereof; wherein said dose provides a once-daily oral dosage of about 400 mg to about 600 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1- yl]azetidin-3-yl}acetonitrile, or a pharmaceutically acceptable salt thereof, to a patient.

The present application further provides one or more sustained release dosage forms as described herein, which together provide a once-daily oral dosage of about 600 mg on a free base basis of {1-{1-[3-fluoro-2-(trifluoromethyl)isonicotinoyl]piperidin-4-y1}-3-[4-(7H-pyrrolo[2,3-d]pyrimidin-4-yl)-1H- pyrazol-1-yl] azetidin-3-yl}acetonitrile, or a pharmaceutically acceptable salt thereof, to a patient.

The present application also provides a dose comprising one or more sustained release dosage forms as described herein, which together provide a once-daily oral dosage of about 600 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof, to a patient.

The present application further provides methods of treating an autoimmune disease, a cancer, a myeloproliferative disorder, an inflammatory disease, a bone resorption disease, or organ transplant rejection in a patient in need thereof, comprising orally administering to said patient one or more sustained release dosage forms as described herein.

The present application also provides methods of treating an autoimmune disease, a cancer, a myeloproliferative disorder, an inflammatory disease, a bone resorption disease, or organ transplant rejection in a patient in need thereof, comprising orally administering to said patient a once-daily dose of about 400 mg to about 600 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof, wherein the dose comprises one or more sustained release dosage forms each comprising {1-{1-[3-fluoro-2-(trifluoromethyl)isonicotinoyl]piperidin-4-yl}-3-[4-(7H-pyrrolo[2,3-d]pyrimidin-4- yl)-1H-pyrazol-1-yl]azetidin-3-yl}acetonitrile, or a pharmaceutically acceptable salt thereof.

The present application further provides methods of treating an autoimmune disease, a cancer, a myeloproliferative disorder, an inflammatory disease, a bone resorption disease, or organ transplant rejection in a patient in need thereof, comprising orally administering to said patient one or more sustained release dosage as described herein.

The present application also provides methods of treating an autoimmune disease, a cancer, a myeloproliferative disorder, an inflammatory disease, a bone resorption disease, or organ transplant rejection in a patient in need thereof, wherein the method comprises orally administering to said patient the one or more sustained release dosage forms as a once-daily dosage of about 600 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof.

### DESCRIPTION OF DRAWINGS

FIG. 1A-C depicts plasma concentrations for the compound of Formula I (Mean±SE) in healthy subjects receiving single doses of 300 mg IR capsules (1A: Cohorts 1-4, fasted), SR1, SR2, SR3, and SR4 tablets (2B: Cohorts 1-4, fasted; and 2C: Cohorts 1-4, fed a high-fat meal).

FIG. 2A-B depicts single-dose 300 mg SR3 PK profiles (Mean±SE) (2A: Cohort 3, SR3, fasted versus high-fat meal; and 2B: Cohort 5, SR3, fasted versus medium-fat meal).

FIG. 3 depicts a comparison of PK profiles (mean±SE) between the 25 mg and 100 mg SR3 tablets (treatment A vs C) and the food effect of a high-fat meal on the 25 mg SR3 tablet (treatment B vs A).

FIG. 4A-B depicts the percent change from baseline for hemoglobin for several dosing regimens for sustained release tablets versus placebo (FIG. 4A as a function of days; FIG. 4B as a function of total average concentration (Cavg)).

FIG. 5A depicts the percentage of patients having a ≥50% reduction in total symptom score (TSS) at week 12 by dose cohort (100 mg BID, 200 mg BID, and 600 mg QD).

FIG. 5B depicts the percent change in total symptom score (TSS) from baseline at week 12 by dose cohort (100 mg BID, 200 mg BID, and 600 mg QD).

FIG. 6A depicts mean hemoglobin levels over time by dose cohort (100 mg BID, 200 mg BID, and 600 mg QD).

FIG. 6B depicts mean hemoglobin levels (g/dL) over time by dose cohort (100 mg BID, 200 mg BID, and 600 mg QD) at 48 weeks.

FIG. 6C depicts mean hemoglobin levels (g/dL) over time by dose cohort at 48 weeks as an average for three dose cohorts as compared to individuals dosed with placebo or ruxolitinib.

### DETAILED DESCRIPTION

The present application provides sustained-release dosage forms comprising {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof. In some embodiments, the present application provides a sustained-release dosage form comprising about 25 mg to about 600 mg on a free base basis of {1-{ 1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof.

In some embodiments, the sustained-release dosage form comprises about 300 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof.

In some embodiments, the sustained-release dosage form comprises about 200 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof.

In some embodiments, the sustained-release dosage form comprises about 100 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof.

In some embodiments, the sustained-release dosage form comprises about 300 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile adipic acid salt.

In some embodiments, the sustained-release dosage form comprises about 200 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl] azetidin-3-yl}acetonitrile adipic acid salt.

In some embodiments, the sustained-release dosage form comprises about 100 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile adipic acid salt.

In some embodiments of the sustained-release dosage form comprising about 100 mg, oral administration of three of said dosage forms to a fasted individual provides a mean peak plasma concentration (Cₘₐₓ) of {1-{1-[3-fluoro-2-(trifluoromethyl)isonicotinoyl]piperidin-4-yl}-3-[4-(7H-pyrrolo[2,3-d]pyrimidin-4- yl)-1H-pyrazol-1-yl]azetidin-3-yl}acetonitrile of about 100 nM to about 1000 nM. As used in this context, oral administration means that a single dose is administered to the individual (in this case, 3×100 mg) and the PK parameter is calculated from the measurements of plasma concentration over time. In this context, the PK parameter (in this case, Cₘₐₓ) is being used to characterize the single sustained release dosage form (i.e., the claims are directed to a single dosage form, not three dosage forms).

In some embodiments of the sustained-release dosage form comprising about 100 mg, oral administration of three of said dosage forms to a fasted individual provides a mean peak plasma concentration (Cₘₐₓ) of {1-{1-[3-fluoro-2-(trifluoromethyl)isonicotinoyl]piperidin-4-yl}-3-[4-(7H-pyrrolo[2,3-d]pyrimidin-4- yl)-1H-pyrazol-1-yl]azetidin-3-yl}acetonitrile of about 400 nM to about 700 nM.

In some embodiments of the sustained-release dosage form comprising about 100 mg, oral administration of three of said dosage forms to a fasted individual provides a mean time to peak plasma concentration (Tₘₐₓ) of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile of about 0.5 hours to about 3 hours.

In some embodiments of the sustained-release dosage form comprising about 100 mg, oral administration of three of said dosage forms to a fasted individual provides a mean time to peak plasma concentration (Tₘₐₓ) of {1-{1-[3-fluoro-2-(trifluoromethyl)isonicotinoyl]piperidin-4-yl}-3-[4-(7H-pyrrolo[2,3-d] pyrimidin-4-yl)-1H-pyrazol-1-yl]azetidin-3-yl}acetonitrile of at least 0.5 hours.

In some embodiments of the sustained-release dosage form comprising about 100 mg, oral administration of three of said dosage forms to a fasted individual provides a ratio of mean peak plasma concentration (Cₘₐₓ) to mean 12-hour plasma concentration (C₁₂ₕ) of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile of about 5 to about 50.

In some embodiments of the sustained-release dosage form comprising about 100 mg, oral administration of three of said dosage forms to a fasted individual provides a ratio of mean peak plasma concentration (Cₘₐₓ) to mean 12-hour plasma concentration (C₁₂ₕ) of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile of about 9 to about 40.

In some embodiments of the sustained-release dosage form comprising about 100 mg, oral administration of three of said dosage forms to a fasted individual provides a ratio of mean peak plasma concentration (Cₘₐₓ) to mean 12-hour plasma concentration (C₁₂ₕ) of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile of about 15 to about 30.

In some embodiments of the sustained-release dosage form comprising about 100 mg, oral administration of three of said dosage forms to a fasted individual provides a mean half-life (t_{1/2}) of {1-{1-[3-fluoro-2-(trifluoromethyl)isonicotinoyl]piperidin-4-yl}-3-[4-(7H-pyrrolo[2,3-d]pyrimidin-4-yl)-1H- pyrazol-1-yl]azetidin-3-yl}acetonitrile of about 5 hours to about 15 hours.

In some embodiments of the sustained-release dosage form comprising about 100 mg, oral administration of three of said dosage forms to a fasted individual provides a mean half-life (t_{1/2}) of {1-{1-[3-fluoro-2-(trifluoromethyl)isonicotinoyl]piperidin-4-yl}-3-[4-(7H-pyrrolo[2,3-d]pyrimidin-4-yl)-1H- pyrazol-1-yl]azetidin-3-yl}acetonitrile of about 7 hours to about 12 hours.

In some embodiments of the sustained-release dosage form comprising about 100 mg, oral administration of three of said dosage forms to a fasted individual provides a mean half-life (t_{1/2}) of {1-{1-[3-fluoro-2-(trifluoromethyl)isonicotinoyl]piperidin-4-yl}-3-[4-(7H-pyrrolo[2,3-d]pyrimidin-4-yl)-1H- pyrazol-1-yl]azetidin-3-yl}acetonitrile of about 1 hour to about 20 hours.

In some embodiments of the sustained-release dosage form comprising about 100 mg, oral administration of three of said dosage forms to a fasted individual provides a mean bioavailability (AUC_{0-∞}) of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile of about 1000 nM*h to about 4000 nM*h.

In some embodiments of the sustained-release dosage form comprising about 100 mg, oral administration of three of said dosage forms to a fasted individual provides a mean bioavailability (AUC_{0-∞}) of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile of about 1500 nM*h to about 3100 nM*h.

In some embodiments of the sustained-release dosage form comprising about 100 mg, oral administration of three of said dosage forms to an individual after a high-fat meal provides a mean peak plasma concentration (Cₘₐₓ) of {1-{1-[3-fluoro-2-(trifluoromethyl)isonicotinoyl]piperidin-4-yl}-3-[4-(7H-pyrrolo[2,3-d] pyrimidin-4-yl)-1H-pyrazol-1-yl]azetidin-3-yl}acetonitrile of about 200 nM to about 2000 nM.

In some embodiments of the sustained-release dosage form comprising about 100 mg, oral administration of three of said dosage forms to an individual after a high-fat meal provides a mean peak plasma concentration (Cₘₐₓ) of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile of about 500 nM to about 1500 nM.

In some embodiments of the sustained-release dosage form comprising about 100 mg, oral administration of three of said dosage forms to an individual after a high-fat meal provides a mean time to peak plasma concentration (Tₘₐₓ) of {1-{1-[3-fluoro-2-(trifluoromethyl)isonicotinoyl]piperidin-4-yl}-3-[4-(7H-pyrrolo[2,3- d]pyrimidin-4-yl)-1H-pyrazol-1-yl]azetidin-3-yl}acetonitrile of about 1 hour to about 9 hours.

In some embodiments of the sustained-release dosage form comprising about 100 mg, oral administration of three of said dosage forms to an individual after a high-fat meal provides a mean time to peak plasma concentration (Tₘₐₓ) of {1-{1-[3-fluoro-2-(trifluoromethyl)isonicotinoyl]piperidin-4-yl}-3-[4-(7H-pyrrolo[2,3- d]pyrimidin-4-yl)-1H-pyrazol-1-yl]azetidin-3-yl}acetonitrile of at least 1.5 hours.

In some embodiments of the sustained-release dosage form comprising about 100 mg, oral administration of three of said dosage forms to an individual after a high-fat meal provides a ratio of mean peak plasma concentration (Cₘₐₓ) to mean 12-hour plasma concentration (C₁₂ₕ) of {1-{1-[3-fluoro-2-(trifluoromethyl)isonicotinoyl] piperidin-4-yl}-3-[4-(7H-pyrrolo[2,3-d]pyrimidin-4-yl)-1H- pyrazol-1-yl]azetidin-3-yl}acetonitrile of about 10 to about 70.

In some embodiments of the sustained-release dosage form comprising about 100 mg, oral administration of three of said dosage forms to an individual after a high-fat meal provides a ratio of mean peak plasma concentration (Cₘₐₓ) to mean 12-hour plasma concentration (C₁₂ₕ) of {1-{1-[3-fluoro-2-(trifluoromethyl)isonicotinoyl]piperidin-4-yl}-3-[4-(7H-pyrrolo[2,3-d]pyrimidin-4-yl)-1H- pyrazol-1-yl]azetidin-3-yl}acetonitrile of about 15 to about 50.

In some embodiments of the sustained-release dosage form comprising about 100 mg, oral administration of three of said dosage forms to an individual after a high-fat meal provides a ratio of mean peak plasma concentration (Cₘₐₓ) to mean 12-hour plasma concentration (C₁₂ₕ) of {1-{1-[3-fluoro-2-(trifluoromethyl)isonicotinoyl]piperidin-4-yl}-3-[4-(7H-pyrrolo[2,3-d]pyrimidin-4-yl)-1H- pyrazol-1-yl]azetidin-3-yl}acetonitrile of about 25 to about 45.

In some embodiments of the sustained-release dosage form comprising about 100 mg, oral administration of three of said dosage forms to an individual after a high-fat meal provides a mean half-life (t_{1/2}) of {1-{1-[3-fluoro-2-(trifluoromethyl) isonicotinoyl]piperidin-4-yl}-3-[4-(7H-pyrrolo[2,3-d]pyrimidin-4-yl)-1H-pyrazol-1-yl]azetidin-3-yl}acetonitrile of about 1 hour to about 7 hours.

In some embodiments of the sustained-release dosage form comprising about 100 mg, oral administration of three of said dosage forms to an individual after a high-fat meal provides a mean half-life (t_{1/2}) of {1-{1-[3-fluoro-2-(trifluoromethyl)isonicotinoyl]piperidin-4-yl}-3-[4-(7H-pyrrolo[2,3-d]pyrimidin-4- yl)-1H-pyrazol-1-yl]azetidin-3-yl}acetonitrile of about 2 hours to about 5 hours.

In some embodiments of the sustained-release dosage form comprising about 100 mg, oral administration of three of said dosage forms to an individual after a high-fat meal provides a mean bioavailability (AUC_{0-∞}) of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile of about 2000 nM*h to about 5000 nM*h.

In some embodiments of the sustained-release dosage form comprising about 100 mg, oral administration of three of said dosage forms to an individual after a high-fat meal provides a mean bioavailability (AUC_{0-%}) of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile of about 3000 nM*h to about 4000 nM*h.

In some embodiments, the percent geometric mean ratio of the sustained release dosage form relative to an immediate release dosage form for Cₘₐₓ is about 15% to about 30%, wherein one or more immediate release dosage forms and one or more sustained release dosage forms are independently orally administered to fasted individuals as a single dose, wherein the same size dose of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt, is administered.

In some embodiments, the percent geometric mean ratio of the sustained release dosage form relative to an immediate release dosage form for Cₘₐₓ is about 15% to about 30%, wherein one or more immediate release dosage forms and one or more sustained release dosage forms are independently orally administered to fasted individuals as a single dose, wherein the same size dose of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt, is administered.

In some embodiments, the percent geometric mean ratio of the sustained release dosage form relative to an immediate release dosage form for AUC_{0-∞} is about 40% to about 55%, wherein one or more immediate release dosage forms and one or more sustained release dosage forms are independently orally administered to fasted individuals as a single dose, wherein the same size dose of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt, is administered.

In some embodiments, the percent geometric mean ratio for Cₘₐₓ of the sustained release dosage form orally administered to an individual after a high-fat meal relative to the sustained release dosage form orally administered to a fasted individual is about 150% to about 250%.

In some embodiments, the percent geometric mean ratio for AUC_{0-∞} of the sustained release dosage form orally administered to an individual after a high-fat meal relative to the sustained release dosage form orally administered to a fasted individual is about 125% to about 170%.

In some embodiments, the sustained-release dosage forms of the invention may include a sustained-release matrix former. Example sustained-release matrix formers include cellulosic ethers such as hydroxypropyl methylcellulose (HPMC, hypromellose) which is a high viscosity polymer, and methyl celluloses. Example hydroxypropyl methylcelluloses include Methocel™ K15M, Methocel™ K4M, Methocel™ K100LV, Methocel™ E3, Methocel™ E5, Methocel™ E6, Methocel™ E15, Methocel™ E50, Methocel™ E10M, Methocel™ E4M, and Methocel™ E10M. In some embodiments, the sustained release dosage form comprises one or more hypromelloses. In some embodiments, the sustained release dosage form comprises a first hypromellose characterized by having an apparent viscosity at a concentration of 2% in water of about 80 cP to about 120 cP and a second hypromellose characterized by having an apparent viscosity at a concentration of 2% in water of about 3000 cP to about 5600 cP. In some embodiments, the sustained release dosage form comprises about 8% to about 20% by weight of one or more hypromelloses. In some embodiments, the sustained release dosage form comprises about 10% to about 15% by weight of one or more hypromelloses.

In some embodiments, the sustained-release dosage forms of the invention can further include one or more fillers, glidants, disintegrants, binders, or lubricants as inactive ingredients. In some embodiments, the filler comprises microcrystalline cellulose, lactose monohydrate, or both. In some embodiments, the sustained release dosage form comprises about 16% to about 22% by weight of microcrystalline cellulose. In some embodiments, the sustained release dosage form comprises about 45% to about 55% by weight of lactose monohydrate.

In some embodiments, lubricants can be present in the dosage forms of the invention in an amount of 0 to about 5% by weight. Non-limiting examples of lubricants include magnesium stearate, stearic acid (stearin), hydrogenated oil, polyethylene glycol, sodium stearyl fumarate, and glyceryl behenate. In some embodiments, the formulations include magnesium stearate, stearic acid, or both. In some embodiments, the sustained release dosage form comprises about 0.3% to about 0.7% by weight of magnesium stearate.

In some embodiments, glidants may be present in the dosage forms. In some embodiments, glidants can be present in the dosage forms of the invention in an amount of 0 to about 5% by weight. Non-limiting examples of glidants include talc, colloidal silicon dioxide, and cornstarch. In some embodiments, the glidant is colloidal silicon dioxide.

In some embodiments, film-coating agents can be present in an amount of 0 to about 5% by weight. Non-limiting illustrative examples of film-coating agents include hypromellose or polyvinyl alcohol based coating with titanium dioxide, talc and optionally colorants available in several commercially available complete coating systems.

In some embodiments, the sustained release dosage form comprises pregelatinized starch.

In some embodiments, the sustained release dosage form is a tablet.

In some embodiments, the sustained release dosage form is prepared by process comprising wet granulation.

In some embodiments, the sustained release dosage form comprises one or more excipients independently selected from hypromelloses and microcrystalline celluloses.

In some embodiments, the sustained release dosage form comprises one or more excipients independently selected from hypromelloses, microcrystalline celluloses, magnesium stearate, lactose, and lactose monohydrate. In some embodiments, the sustained release dosage form comprises one or more excipients independently selected from hypromelloses, microcrystalline celluloses, magnesium stearate, lactose, lactose monohydrate, and pregelatinized starch. The present invention further provides one or more sustained release dosage forms each comprising {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof; wherein said one or more sustained release dosage forms together provide a once-daily oral dosage of about 400 mg to about 600 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof, to a patient.

The present invention also provides a dose, comprising one or more sustained release dosage forms each comprising {1-{1-[3-fluoro-2-(trifluoromethyl)isonicotinoyl]piperidin-4-yl}-3-[4-(7H-pyrrolo[2,3-d]pyrimidin-4-yl)-1H- pyrazol-1-yl]azetidin-3-yl}acetonitrile, or a pharmaceutically acceptable salt thereof; wherein said dose provides a once-daily oral dosage of about 400 mg to about 600 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1- yl]azetidin-3-yl}acetonitrile, or a pharmaceutically acceptable salt thereof, to a patient.

The present application further provides one or more sustained release dosage forms as described herein, which together provide a once-daily oral dosage of about 600 mg on a free base basis of {1-{1-[3-fluoro-2-(trifluoromethyl)isonicotinoyl]piperidin-4-y1}-3-[4-(7H-pyrrolo[2,3-d]pyrimidin-4-yl)-1H- pyrazol-1-yl]azetidin-3-yl}acetonitrile, or a pharmaceutically acceptable salt thereof, to a patient.

The present application further provides one or more sustained release dosage forms as described herein, which together provide a once-daily oral dosage of about 500 mg on a free base basis of {1-{1-[3-fluoro-2-(trifluoromethyl)isonicotinoyl]piperidin-4-y1}-3-[4-(7H-pyrrolo[2,3-d]pyrimidin-4-yl)-1H- pyrazol-1-yl]azetidin-3-yl}acetonitrile, or a pharmaceutically acceptable salt thereof, to a patient.

The present application further provides one or more sustained release dosage forms as described herein, which together provide a once-daily oral dosage of about 400 mg on a free base basis of {1-{1-[3-fluoro-2-(trifluoromethyl)isonicotinoyl]piperidin-4-y1}-3-[4-(7H-pyrrolo[2,3-d]pyrimidin-4-yl)-1H- pyrazol-1-yl]azetidin-3-yl}acetonitrile, or a pharmaceutically acceptable salt thereof, to a patient.

In some embodiments, the one or more sustained release dosage forms are six dosage forms of about 100 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof, are provided. In some embodiments, the one or more sustained release dosage forms are three dosage forms of about 200 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof, are provided. In some embodiments, the one or more sustained release dosage forms are two dosage forms of about 300 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof, are provided. In some embodiments, the one or more sustained release dosage forms is one dosage form of about 600 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof, is provided.

The present application also provides a dose comprising one or more sustained release dosage forms as described herein, which provide a once-daily oral dosage of about 600 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof, to a patient.

The present application also provides a dose comprising one or more sustained release dosage forms as described herein, which provide a once-daily oral dosage of about 500 mg on a free base basis of {1-{1-[3-fluoro-2-(trifluoromethyl)isonicotinoyl]piperidin-4-yl}-3-[4-(7H-pyrrolo[2,3-d] pyrimidin-4-yl)- 1H-pyrazol-1-yl]azetidin-3-yl}acetonitrile, or a pharmaceutically acceptable salt thereof, to a patient.

The present application also provides a dose comprising one or more sustained release dosage forms as described herein, which provide a once-daily oral dosage of about 400 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof, to a patient.

In some embodiments, the dose comprises six dosage forms of about 100 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1- yl]azetidin-3-yl}acetonitrile, or a pharmaceutically acceptable salt thereof. In some embodiments, the dose comprises three dosage forms of about 200 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof. In some embodiments, the dose comprises two dosage forms of about 300 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof. In some embodiments, the dose comprises one dosage form of about 600 mg on a free base basis of {1-{1-[3-fluoro-2-(trifluoromethyl) isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof.

The present application further provides a kit comprising one or more sustained release dosage forms as described herein, which together provide a once-daily oral dosage of about 400 mg to about 600 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof, to a patient. In some embodiments, the kit further comprises an instruction to administer the one or more sustained release dosage forms as a once-daily dose of about 400 mg to about 600 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof.

The present application further provides a kit comprising one or more sustained release dosage forms as described herein, which together provide a once-daily oral dosage of about 600 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof, to a patient. In some embodiments, the kit further comprises an instruction to administer the one or more sustained release dosage forms as a once-daily dose of about 600 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1- yl]azetidin-3-yl}acetonitrile, or a pharmaceutically acceptable salt thereof.

The present application further provides a kit comprising one or more sustained release dosage forms as described herein, which together provide a once-daily oral dosage of about 500 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof, to a patient. In some embodiments, the kit further comprises an instruction to administer the one or more sustained release dosage forms as a once-daily dose of about 600 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1- yl]azetidin-3-yl}acetonitrile, or a pharmaceutically acceptable salt thereof.

The present application further provides a kit comprising one or more sustained release dosage forms as described herein, which together provide a once-daily oral dosage of about 400 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof, to a patient. In some embodiments, the kit further comprises an instruction to administer the one or more sustained release dosage forms as a once-daily dose of about 600 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1- yl]azetidin-3-yl}acetonitrile, or a pharmaceutically acceptable salt thereof.

In some embodiments, the kit comprises six dosage forms of about 100 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1- yl]azetidin-3-yl}acetonitrile, or a pharmaceutically acceptable salt thereof. In some embodiments, the kit comprises three dosage forms of about 200 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof. In some embodiments, the kit comprises two dosage forms of about 300 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof. In some embodiments, the kit comprises one dosage form of about 600 mg on a free base basis of {1-{1-[3-fluoro-2-(trifluoromethyl)isonicotinoyl]piperidin-4-yl}-3-[4-(7H-pyrrolo[2,3-d]pyrimidin- 4-yl)-1H-pyrazol-1-yl]azetidin-3-yl}acetonitrile, or a pharmaceutically acceptable salt thereof.

As used herein, “sustained-release” is used as generally understood in the art and refers to a formulation designed to slowly release the active ingredient into a patient after oral administration.

As used herein, “dose” refers to the total amount of the compound of Formula I orally administered to the individual or patient. The dose may be in a single dosage form, or a plurality of dosage forms (e.g., a 600 mg dose may be one 600 mg dosage form, two 300 mg dosage forms, three 200 mg dosage forms, six 100 mg dosage forms, etc.). Hence, a dose can refer to a plurality of pills to be taken by a patient at nearly simultaneously.

As used herein, “a fasted individual” means an individual who has fasted for at least 10 hours prior to administration of the dose.

As used herein, “mean” when preceding a pharmacokinetic value (e.g. mean Cₘₐₓ) represents the arithmetic mean value of the pharmacokinetic value taken from a population of patients unless otherwise specified.

As used herein, “Cₘₐₓ” means the maximum observed plasma concentration.

As used herein, “C₁₂ₕ” refers to the plasma concentration measured at 12 hours from administration.

As used herein, “Tₘₐₓ” refers to the time at which the maximum blood plasma concentration is observed.

As used herein, “T_{1/2}” refers to the time at which the plasma concentration is half of the observed maximum.

As used herein, “AUC” refers to the area under the plasma concentration-time curve which is a measure of total bioavailability.

As used herein, “AUC_{0-∞}” refers to the area under the plasma concentration-time curve extrapolated to infinity.

As used herein, “AUC₀₋ₜ” refers to the area under the plasma concentration-time curve from time 0 to the last time point with a quantifiable plasma concentration, usually about 12-36 hours.

As used herein, “AUC_{0-τ}” refers to the area under the plasma concentration-time curve from time 0 to the time of the next dose.

As used herein, “Cl/F” refers to oral clearance.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety. In some embodiments, the compounds described herein include the N-oxide forms.

### Methods

The present application further provides methods of treating an autoimmune disease, a cancer, a myeloproliferative disorder, an inflammatory disease, a bone resorption disease, or organ transplant rejection in a patient in need thereof, comprising orally administering to said patient one or more sustained release dosage forms as described herein.

The present application also provides a method of treating an autoimmune disease, a cancer, a myeloproliferative disorder, an inflammatory disease, a bone resorption disease, or organ transplant rejection in a patient in need thereof, comprising orally administering to said patient a once-daily dose of about 400 mg to about 600 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof, wherein the dose comprises one or more sustained release dosage forms each comprising {1-{1-[3-fluoro-2-(trifluoromethyl)isonicotinoyl]piperidin-4-yl}-3-[4-(7H-pyrrolo[2,3-d]pyrimidin-4- yl)-1H-pyrazol-1-yl]azetidin-3-yl}acetonitrile, or a pharmaceutically acceptable salt thereof.

The present application further provides a method of treating an autoimmune disease, a cancer, a myeloproliferative disorder, an inflammatory disease, a bone resorption disease, or organ transplant rejection in a patient in need thereof, comprising orally administering to said patient one or more sustained release dosage as described herein.

The present application also provides a method of treating an autoimmune disease, a cancer, a myeloproliferative disorder, an inflammatory disease, a bone resorption disease, or organ transplant rejection in a patient in need thereof, wherein the method comprises orally administering to said patient the one or more sustained release dosage forms as a once-daily dosage of about 600 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof.

The present application also provides a method of treating an autoimmune disease, a cancer, a myeloproliferative disorder, an inflammatory disease, a bone resorption disease, or organ transplant rejection in a patient in need thereof, wherein the method comprises orally administering to said patient the one or more sustained release dosage forms as a once-daily dosage of about 500 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof.

The present application also provides a method of treating an autoimmune disease, a cancer, a myeloproliferative disorder, an inflammatory disease, a bone resorption disease, or organ transplant rejection in a patient in need thereof, wherein the method comprises orally administering to said patient the one or more sustained release dosage forms as a once-daily dosage of about 400 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof.

In some embodiments of the methods in the preceding three paragraphs, the one or more sustained release dosage forms are six dosage forms of about 100 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof, are provided. In some embodiments of the methods in the preceding three paragraphs, the one or more sustained release dosage forms are three dosage forms of about 200 mg on a free base basis of {1-{1-[3-fluoro-2-(trifluoromethyl) isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof, are provided. In some embodiments of the methods in the preceding three paragraphs, the one or more sustained release dosage forms are two dosage forms of about 300 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1- yl]azetidin-3-yl}acetonitrile, or a pharmaceutically acceptable salt thereof, are provided. In some embodiments of the methods in the preceding three paragraphs, the one or more sustained release dosage forms is one dosage form of about 600 mg on a free base basis of {1-{1-[3-fluoro-2-(trifluoromethyl)isonicotinoyl]piperidin-4-yl}-3-[4-(7H-pyrrolo[2,3- d]pyrimidin-4-yl)-1H-pyrazol-1-yl]azetidin-3-yl}acetonitrile, or a pharmaceutically acceptable salt thereof, is provided.

In some embodiments, oral administration of one or more sustained release dosage forms to a fasted individual provides a mean time to peak plasma concentration (Tₘₐₓ) of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile of about 0.5hours to about 3 hours.

In some embodiments, oral administration of one or more sustained release dosage forms to a fasted individual provides a mean time to peak plasma concentration (Tₘₐₓ) of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile of at least 0.5 hours.

In some embodiments, oral administration of one or more sustained release dosage forms to a fasted individual provides a ratio of mean peak plasma concentration (Cₘₐₓ) to mean 12-hour plasma concentration (C₁₂ₕ) of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile of about 5 to about 50.

In some embodiments, oral administration of one or more sustained release dosage forms to a fasted individual provides a ratio of mean peak plasma concentration (Cₘₐₓ) to mean 12-hour plasma concentration (C₁₂ₕ) of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile of about 9 to about 40.

In some embodiments, oral administration of one or more sustained release dosage forms to a fasted individual provides a ratio of mean peak plasma concentration (Cₘₐₓ) to mean 12-hour plasma concentration (C₁₂ₕ) of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile of about 15 to about 30.

In some embodiments, oral administration of one or more sustained release dosage forms to a fasted individual provides a mean half-life (t_{1/2}) of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile of about 1 hour to about 20 hours.

In some embodiments, oral administration of one or more sustained release dosage forms to an individual after a high-fat meal provides a mean time to peak plasma concentration (Tₘₐₓ) of {1-{1-[3-fluoro-2-(trifluoromethyl)isonicotinoyl]piperidin-4-yl}-3-[4-(7H-pyrrolo[2,3-d]pyrimidin-4-yl)-1H- pyrazol-1-yl]azetidin-3-yl}acetonitrile of about 1 hour to about 9 hours.

In some embodiments, oral administration of one or more sustained release dosage forms to an individual after a high-fat meal provides a mean time to peak plasma concentration (Tₘₐₓ) of {1-{1-[3-fluoro-2-(trifluoromethyl)isonicotinoyl]piperidin-4-yl}-3-[4-(7H-pyrrolo[2,3-d]pyrimidin-4-yl)-1H- pyrazol-1-yl]azetidin-3-yl}acetonitrile of at least 1.5 hours.

In some embodiments, oral administration of one or more sustained release dosage forms to an individual after a high-fat meal provides a ratio of mean peak plasma concentration (Cₘₐₓ) to mean 12-hour plasma concentration (C₁₂ₕ) of {1-{1-[3-fluoro-2-(trifluoromethyl)isonicotinoyl]piperidin-4-yl}-3-[4-(7H- pyrrolo[2,3-d]pyrimidin-4-yl)-1H-pyrazol-1-yl]azetidin-3-yl}acetonitrile of about 10 to about 70.

In some embodiments, oral administration of one or more sustained release dosage forms to an individual after a high-fat meal provides a ratio of mean peak plasma concentration (Cₘₐₓ) to mean 12-hour plasma concentration (C₁₂ₕ) of {1-{ 1-[3-fluoro-2-(trifluoromethyl)isonicotinoyl]piperidin-4-yl}-3-[4-(7H- pyrrolo[2,3-d]pyrimidin-4-yl)-1H-pyrazol-1-yl]azetidin-3-yl}acetonitrile of about 15 to about 50.

In some embodiments, oral administration of one or more sustained release dosage forms to an individual after a high-fat meal provides a ratio of mean peak plasma concentration (Cₘₐₓ) to mean 12-hour plasma concentration (C₁₂ₕ) of {1-{1-[3-fluoro-2-(trifluoromethyl)isonicotinoyl]piperidin-4-yl}-3-[4-(7H- pyrrolo[2,3-d]pyrimidin-4-yl)-1H-pyrazol-1-yl]azetidin-3-yl}acetonitrile of about 25 to about 45.

In some embodiments, oral administration of one or more sustained release dosage forms to an individual after a high-fat meal provides a mean half-life (t_{1/2}) of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile of about 1 hour to about 7 hours.

In some embodiments, oral administration of one or more sustained release dosage forms to an individual after a high-fat meal provides a mean half-life (t_{1/2}) of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile of about 2 hours to about 5 hours.

In some embodiments, the one or more sustained release dosage forms are each a tablet. In some embodiments, the one or more sustained release dosage forms are prepared by process comprising wet granulation.

In some embodiments, the one or more sustained release dosage forms each comprises one or more hypromelloses. In some embodiments, the one or more sustained release dosage forms each comprises one or more excipients independently selected from hypromelloses and microcrystalline celluloses. In some embodiments, the one or more sustained release dosage forms each comprises one or more excipients independently selected from hypromelloses, microcrystalline celluloses, magnesium stearate, lactose, and lactose monohydrate. In some embodiments, the one or more sustained release dosage forms each comprises a first hypromellose characterized by having an apparent viscosity at a concentration of 2% in water of about 80 cP to about 120 cP and a second hypromellose characterized by having an apparent viscosity at a concentration of 2% in water of about 3000 cP to about 5600 cP.

In some embodiments, the one or more sustained release dosage forms each comprises about 10% to about 15% by weight of one or more hypromelloses. In some embodiments, the one or more sustained release dosage forms each comprises about 16% to about 22% by weight of microcrystalline cellulose. In some embodiments, the one or more sustained release dosage forms each comprises about 45% to about 55% by weight of lactose monohydrate. In some embodiments, the one or more sustained release dosage forms each comprises about 0.3% to about 0.7% by weight of magnesium stearate.

In some embodiments, the present application provides a method of treating myelofibrosis in a patient, comprising orally administering to said patient a once-daily dose of about 400 mg to about 600 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof, wherein the dose comprises one or more sustained release dosage forms each comprising {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl] azetidin-3-yl}acetonitrile, or a pharmaceutically acceptable salt thereof; wherein the method results in a reduced total symptom score (TSS) of said patient compared with baseline. In some embodiments, the present application provides a method of treating myelofibrosis in a patient, comprising orally administering to said patient the one or more sustained release dosage forms as a once-daily dosage of about 600 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof, wherein the method results in a reduced total symptom score (TSS) of said patient compared with baseline.

In some embodiments, the present application provides a method of treating myelofibrosis in a patient, comprising orally administering to said patient the one or more sustained release dosage forms as a once-daily dosage of about 500 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof; wherein the method results in a reduced total symptom score (TSS) of said patient compared with baseline.

In some embodiments, the present application provides a method of treating myelofibrosis in a patient, comprising orally administering to said patient the one or more sustained release dosage forms as a once-daily dosage of about 400 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof; wherein the method results in a reduced total symptom score (TSS) of said patient compared with baseline.

In some embodiments of the methods in the preceding three paragraphs, the one or more sustained release dosage forms are six dosage forms of about 100 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof, are provided. In some embodiments of the methods in the preceding three paragraphs, the one or more sustained release dosage forms are three dosage forms of about 200 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof, are provided. In some embodiments of the methods in the preceding three paragraphs, the one or more sustained release dosage forms are two dosage forms of about 300 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1- yl]azetidin-3-yl}acetonitrile, or a pharmaceutically acceptable salt thereof, are provided. In some embodiments of the methods in the preceding three paragraphs, the one or more sustained release dosage forms is one dosage form of about 600 mg on a free base basis of {1-{1-[3-fluoro-2-(trifluoromethyl)isonicotinoyl]piperidin-4-yl}-3-[4-(7H-pyrrolo[2,3- d]pyrimidin-4-yl)-1H-pyrazol-1-yl]azetidin-3-yl}acetonitrile, or a pharmaceutically acceptable salt thereof, is provided.

In some embodiments, “total symptom score (TSS)” refers to the TSS derived from the modified Myelofibrosis Symptom Assessment Form (MFSAF) (e.g., v3.0) electronic diary as compared with baseline (baseline is the patient's baseline TSS before treatment). In some embodiments, myelofibrosis is primary myelofibrosis (PMF), post-polycythemia vera MF, or post-essential thrombocythemia MF.

The present application also provides a method of treating an autoimmune disease, a cancer, a myeloproliferative disorder, an inflammatory disease, a bone resorption disease, or organ transplant rejection in a patient in need thereof, comprising orally administering to said patient a once-daily dose of about 400 mg to about 600 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof, wherein the dose comprises one or more sustained release dosage forms each comprising {1-{1-[3-fluoro-2-(trifluoromethyl)isonicotinoyl]piperidin-4-yl}-3-[4-(7H-pyrrolo[2,3-d]pyrimidin-4- yl)-1H-pyrazol-1-yl]azetidin-3-yl}acetonitrile, or a pharmaceutically acceptable salt thereof; wherein said method results in reduced anemia.

The present application also provides a method of treating an autoimmune disease, a cancer, a myeloproliferative disorder, an inflammatory disease, a bone resorption disease, or organ transplant rejection in a patient in need thereof, wherein the method comprises orally administering to said patient the one or more sustained release dosage forms as a once-daily dosage of about 600 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof; wherein said method results in reduced anemia.

The present application also provides a method of treating an autoimmune disease, a cancer, a myeloproliferative disorder, an inflammatory disease, a bone resorption disease, or organ transplant rejection in a patient in need thereof, wherein the method comprises orally administering to said patient the one or more sustained release dosage forms as a once-daily dosage of about 500 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof; wherein said method results in reduced anemia.

The present application also provides a method of treating an autoimmune disease, a cancer, a myeloproliferative disorder, an inflammatory disease, a bone resorption disease, or organ transplant rejection in a patient in need thereof, wherein the method comprises orally administering to said patient the one or more sustained release dosage forms as a once-daily dosage of about 400 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof; wherein said method results in reduced anemia. In some embodiments, the one or more sustained release dosage forms are six dosage forms of about 100 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof, are provided. In some embodiments, the one or more sustained release dosage forms are three dosage forms of about 200 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof, are provided. In some embodiments, the one or more sustained release dosage forms are two dosage forms of about 300 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof, are provided. In some embodiments, the one or more sustained release dosage forms is one dosage form of about 600 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof, is provided.

Reduced anemia is relative to that experienced for a twice-daily dose of 200 mg on a free base basis of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof, wherein the dose comprises one or more sustained release dosage forms each comprising {1-{1-[3-fluoro-2-(trifluoromethyl)isonicotinoyl]piperidin-4-yl}-3-[4-(7H-pyrrolo[2,3-d]pyrimidin-4- yl)-1H-pyrazol-1-yl]azetidin-3-yl}acetonitrile, or a pharmaceutically acceptable salt thereof.

The compound of Formula I is a JAK inhibitor. A JAK1 selective inhibitor is a compound that inhibits JAK1 activity preferentially over other Janus kinases. JAK1 plays a central role in a number of cytokine and growth factor signaling pathways that, when dysregulated, can result in or contribute to disease states. For example, IL-6 levels are elevated in rheumatoid arthritis, a disease in which it has been suggested to have detrimental effects (Fonesca, J. E. et al., Autoimmunity Reviews, 8:538-42, 2009). Because IL-6 signals, at least in part, through JAK1, antagonizing IL-6 directly or indirectly through JAK1 inhibition is expected to provide clinical benefit (Guschin, D., N., et al Embo J 14:1421, 1995; Smolen, J. S., et al. Lancet 371:987, 2008). Moreover, in some cancers JAK1 is mutated resulting in constitutive undesirable tumor cell growth and survival (Mullighan C G, Proc Natl Acad Sci U S A. 106:9414-8, 2009; Flex E., et al. J Exp Med. 205:751-8, 2008). In other autoimmune diseases and cancers elevated systemic levels of inflammatory cytokines that activate JAK1 may also contribute to the disease and/or associated symptoms. Therefore, patients with such diseases may benefit from JAK1 inhibition. Selective inhibitors of JAK1 may be efficacious while avoiding unnecessary and potentially undesirable effects of inhibiting other JAK kinases.

Selective inhibitors of JAK1, relative to other JAK kinases, may have multiple therapeutic advantages over less selective inhibitors. With respect to selectivity against JAK2, a number of important cytokines and growth factors signal through JAK2 including, for example, erythropoietin (Epo) and thrombopoietin (Tpo) (Parganas E, et al. Cell. 93:385-95, 1998). Epo is a key growth factor for red blood cells production; hence a paucity of Epo-dependent signaling can result in reduced numbers of red blood cells and anemia (Kaushansky K, NEJM 354:2034-45, 2006). Tpo, another example of a JAK2-dependent growth factor, plays a central role in controlling the proliferation and maturation of megakaryocytes-the cells from which platelets are produced (Kaushansky K, NEJM 354:2034-45, 2006). As such, reduced Tpo signaling would decrease megakaryocyte numbers (megakaryocytopenia) and lower circulating platelet counts (thrombocytopenia). This can result in undesirable and/or uncontrollable bleeding. Reduced inhibition of other JAKs, such as JAK3 and Tyk2, may also be desirable as humans lacking functional version of these kinases have been shown to suffer from numerous maladies such as severe-combined immunodeficiency or hyperimmunoglobulin E syndrome (Minegishi, Y, et al. Immunity 25:745-55, 2006; Macchi P, et al. Nature. 377:65-8, 1995). Therefore a JAK1 inhibitor with reduced affinity for other JAKs would have significant advantages over a less-selective inhibitor with respect to reduced side effects involving immune suppression, anemia and thrombocytopenia.

Another aspect of the present invention pertains to methods of treating a JAK-associated disease or disorder in an individual (e.g., patient) by administering to the individual in need of such treatment a sustained-release dosage form of the invention. A JAK-associated disease can include any disease, disorder or condition that is directly or indirectly linked to expression or activity of the JAK, including overexpression and/or abnormal activity levels. A JAK-associated disease can also include any disease, disorder or condition that can be prevented, ameliorated, or cured by modulating JAK activity.

Examples of JAK-associated diseases include diseases involving the immune system including, for example, organ transplant rejection (e.g., allograft rejection and graft versus host disease).

Further examples of JAK-associated diseases include autoimmune diseases such as multiple sclerosis, rheumatoid arthritis, juvenile arthritis, psoriatic arthritis, type I diabetes, lupus, psoriasis, inflammatory bowel disease, ulcerative colitis, Crohn's disease, myasthenia gravis, immunoglobulin nephropathies, myocarditis, autoimmune thyroid disorders, chronic obstructive pulmonary disease (COPD), and the like. In some embodiments, the autoimmune disease is an autoimmune bullous skin disorder such as pemphigus vulgaris (PV) or bullous pemphigoid (BP).

Further examples of JAK-associated diseases include allergic conditions such as asthma, food allergies, eszematous dermatitis, contact dermatitis, atopic dermatitis (atropic eczema), and rhinitis. Further examples of JAK-associated diseases include viral diseases such as Epstein Barr Virus (EBV), Hepatitis B, Hepatitis C, HIV, HTLV 1, Varicella-Zoster Virus (VZV) and Human Papilloma Virus (HPV). Further examples of JAK-associated disease include diseases associated with cartilage turnover, for example, gouty arthritis, septic or infectious arthritis, reactive arthritis, reflex sympathetic dystrophy, algodystrophy, Tietze syndrome, costal athropathy, osteoarthritis deformans endemica, Mseleni disease, Handigodu disease, degeneration resulting from fibromyalgia, systemic lupus erythematosus, scleroderma, or ankylosing spondylitis.

Further examples of JAK-associated disease include congenital cartilage malformations, including hereditary chrondrolysis, chrondrodysplasias, and pseudochrondrodysplasias (e.g., microtia, enotia, and metaphyseal chrondrodysplasia).

Further examples of JAK-associated diseases or conditions include skin disorders such as psoriasis (for example, psoriasis vulgaris), atopic dermatitis, skin rash, skin irritation, skin sensitization (e.g., contact dermatitis or allergic contact dermatitis). For example, certain substances including some pharmaceuticals when topically applied can cause skin sensitization. In some embodiments, co-administration or sequential administration of at least one JAK inhibitor of the invention together with the agent causing unwanted sensitization can be helpful in treating such unwanted sensitization or dermatitis. In some embodiments, the skin disorder is treated by topical administration of at least one JAK inhibitor of the invention.

In further embodiments, the JAK-associated disease is cancer including those characterized by solid tumors (e.g., prostate cancer, renal cancer, hepatic cancer, pancreatic cancer, gastric cancer, breast cancer, lung cancer, cancers of the head and neck, thyroid cancer, glioblastoma, Kaposi's sarcoma, Castleman's disease, uterine leiomyosarcoma, melanoma etc.), hematological cancers (e.g., lymphoma, leukemia such as acute lymphoblastic leukemia (ALL), acute myelogenous leukemia (AML) or multiple myeloma), and skin cancer such as cutaneous T-cell lymphoma (CTCL) and cutaneous B-cell lymphoma. Example CTCLs include Sezary syndrome and mycosis fungoides.

In some embodiments, the dosage forms described herein, or in combination with other JAK inhibitors, such as those reported in U.S. Ser. No. 11/637,545, which is incorporated herein by reference in its entirety, can be used to treat inflammation-associated cancers. In some embodiments, the cancer is associated with inflammatory bowel disease. In some embodiments, the inflammatory bowel disease is ulcerative colitis. In some embodiments, the inflammatory bowel disease is Crohn's disease. In some embodiments, the inflammation-associated cancer is colitis-associated cancer. In some embodiments, the inflammation-associated cancer is colon cancer or colorectal cancer. In some embodiments, the cancer is gastric cancer, gastrointestinal carcinoid tumor, gastrointestinal stromal tumor (GIST), adenocarcinoma, small intestine cancer, or rectal cancer.

JAK-associated diseases can further include those characterized by expression of: JAK2 mutants such as those having at least one mutation in the pseudo-kinase domain (e.g., JAK2V617F); JAK2 mutants having at least one mutation outside of the pseudo-kinase domain; JAK1 mutants; JAK3 mutants; erythropoietin receptor (EPOR) mutants; or deregulated expression of CRLF2.

JAK-associated diseases can further include myeloproliferative disorders (MPDs) such as polycythemia vera (PV), essential thrombocythemia (ET), myelofibrosis with myeloid metaplasia (MMM), primary myelofibrosis (PMF), chronic myelogenous leukemia (CML), chronic myelomonocytic leukemia (CMML), hypereosinophilic syndrome (HES), systemic mast cell disease (SMCD), and the like. In some embodiments, the myeloproliferative disorder is myelofibrosis (e.g., primary myelofibrosis (PMF) or post polycythemia vera/essential thrombocythemia myelofibrosis (Post-PV/ET MF)). In some embodiments, the myeloproliferative disorder is post-essential thrombocythemia myelofibrosis (Post-ET). In some embodiments, the myeloproliferative disorder is post polycythemia vera myelofibrosis (Post-PV MF).

In some embodiments, dosage forms described herein can be used to treat pulmonary arterial hypertension.

The present invention further provides a method of treating dermatological side effects of other pharmaceuticals by administration of the dosage forms of the invention. For example, numerous pharmaceutical agents result in unwanted allergic reactions which can manifest as acneiform rash or related dermatitis. Example pharmaceutical agents that have such undesirable side effects include anti-cancer drugs such as gefitinib, cetuximab, erlotinib, and the like. The dosage forms of the invention can be administered systemically in combination with (e.g., simultaneously or sequentially) the pharmaceutical agent having the undesirable dermatological side effect.

Further JAK-associated diseases include inflammation and inflammatory diseases. Example inflammatory diseases include sarcoidosis, inflammatory diseases of the eye (e.g., iritis, uveitis, scleritis, conjunctivitis, or related disease), inflammatory diseases of the respiratory tract (e.g., the upper respiratory tract including the nose and sinuses such as rhinitis or sinusitis or the lower respiratory tract including bronchitis, chronic obstructive pulmonary disease, and the like), inflammatory myopathy such as myocarditis, and other inflammatory diseases. In some embodiments, the inflammation disease of the eye is blepharitis.

The dosage forms described herein can further be used to treat ischemia reperfusion injuries or a disease or condition related to an inflammatory ischemic event such as stroke or cardiac arrest. The dosage forms described herein can further be used to treat endotoxin-driven disease state (e.g., complications after bypass surgery or chronic endotoxin states contributing to chronic cardiac failure). The dosage forms described herein can further be used to treat anorexia, cachexia, or fatigue such as that resulting from or associated with cancer. The dosage forms described herein can further be used to treat restenosis, sclerodermitis, or fibrosis. The dosage forms described herein can further be used to treat conditions associated with hypoxia or astrogliosis such as, for example, diabetic retinopathy, cancer, or neurodegeneration. See, e.g., Dudley, A. C. et al. Biochem. J. 2005, 390 (Pt 2): 427-36 and Sriram, K. et al. J. Biol. Chem. 2004, 279(19): 19936-47. Epub 2004 Mar. 2, both of which are incorporated herein by reference in their entirety. The JAK inhibitors described herein can be used to treat Alzheimer's disease.

The dosage forms described herein can further be used to treat other inflammatory diseases such as systemic inflammatory response syndrome (SIRS) and septic shock.

The dosage forms described herein can further be used to treat gout and increased prostate size due to, e.g., benign prostatic hypertrophy or benign prostatic hyperplasia. Further JAK-associated diseases include bone resorption diseases such as osteoporosis, osteoarthritis. Bone resorption can also be associated with other conditions such as hormonal imbalance and/or hormonal therapy, autoimmune disease (e.g. osseous sarcoidosis), or cancer (e.g. myeloma). The reduction of the bone resorption due to the the compound of Formula I can be about 10%, about 20%, about 30%, about 40%, about 50%, about 60%, about 70%, about 80%, or about 90%.

In some embodiments, the dosage forms described herein can further be used to treat a dry eye disorder. As used herein, “dry eye disorder” is intended to encompass the disease states summarized in a recent official report of the Dry Eye Workshop (DEWS), which defined dry eye as “a multifactorial disease of the tears and ocular surface that results in symptoms of discomfort, visual disturbance, and tear film instability with potential damage to the ocular surface. It is accompanied by increased osmolarity of the tear film and inflammation of the ocular surface.” Lemp, “The Definition and Classification of Dry Eye Disease: Report of the Definition and Classification Subcommittee of the International Dry Eye Workshop”, The Ocular Surface, 5(2), 75-92 April 2007, which is incorporated herein by reference in its entirety. In some embodiments, the dry eye disorder is selected from aqueous tear-deficient dry eye (ADDE) or evaporative dry eye disorder, or appropriate combinations thereof. In some embodiments, the dry eye disorder is Sjogren syndrome dry eye (SSDE). In some embodiments, the dry eye disorder is non-Sjogren syndrome dry eye (NSSDE).

In a further aspect, the present invention provides a method of treating conjunctivitis, uveitis (including chronic uveitis), chorioditis, retinitis, cyclitis, sclieritis, episcleritis, or iritis; treating inflammation or pain related to corneal transplant, LASIK (laser assisted in situ keratomileusis), photorefractive keratectomy, or LASEK (laser assisted sub-epithelial keratomileusis); inhibiting loss of visual acuity related to corneal transplant, LASIK, photorefractive keratectomy, or LASEK; or inhibiting transplant rejection in a patient in need thereof, comprising administering to the patient a dosage form of the invention.

Additionally, the dosage forms of the invention, or in combination with other JAK inhibitors, such as those reported in U.S. Ser. No. 11/637,545, which is incorporated herein by reference in its entirety, can be used to treat respiratory dysfunction or failure associated with viral infection, such as influenza and SARS.

In some embodiments, the present invention provides a dosage form as described in any of the embodiments herein, for use in a method of treating any of the diseases or disorders described herein. In some embodiments, the present invention provides the use of a dosage form as described in any of the embodiments herein, for the preparation of a medicament for use in a method of treating any of the diseases or disorders described herein.

In some embodiments, the present invention provides a dosage form as described herein, or a pharmaceutically acceptable salt thereof, for use in a method of modulating JAK1. In some embodiments, the present invention also provides use of a dosage form as described herein, or a pharmaceutically acceptable salt thereof, for the preparation of a medicament for use in a method of modulating JAK1.

As used herein, the term “individual” is a human. In some embodiments, the human is an adult subject.

As used herein, the term “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); and (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

### Combination Therapies

One or more additional pharmaceutical agents such as, for example, chemotherapeutics, anti-inflammatory agents, steroids, immunosuppressants, as well as Bcr-Abl, Flt-3, RAF and FAK kinase inhibitors such as, for example, those described in WO 2006/056399, which is incorporated herein by reference in its entirety, or other agents can be used in combination with the dosage forms described herein for treatment of JAK-associated diseases, disorders or conditions. The one or more additional pharmaceutical agents can be administered to a patient simultaneously or sequentially.

Example chemotherapeutics include proteosome inhibitors (e.g., bortezomib), thalidomide, revlimid, and DNA-damaging agents such as melphalan, doxorubicin, cyclophosphamide, vincristine, etoposide, carmustine, and the like.

Example steroids include coriticosteroids such as dexamethasone or prednisone.

Example Bcr-Abl inhibitors include the compounds, and pharmaceutically acceptable salts thereof, of the genera and species disclosed in U.S. Pat. No. 5,521,184, WO 04/005281, and U.S. Ser. No. 60/578,491, all of which are incorporated herein by reference in their entirety.

Example suitable Flt-3 inhibitors include compounds, and their pharmaceutically acceptable salts, as disclosed in WO 03/037347, WO 03/099771, and WO 04/046120, all of which are incorporated herein by reference in their entirety.

Example suitable RAF inhibitors include compounds, and their pharmaceutically acceptable salts, as disclosed in WO 00/09495 and WO 05/028444, both of which are incorporated herein by reference in their entirety.

Example suitable FAK inhibitors include compounds, and their pharmaceutically acceptable salts, as disclosed in WO 04/080980, WO 04/056786, WO 03/024967, WO 01/064655, WO 00/053595, and WO 01/014402, all of which are incorporated herein by reference in their entirety.

In some embodiments, one or more of the dosage forms of the invention can be used in combination with one or more other kinase inhibitors including imatinib, particularly for treating patients resistant to imatinib or other kinase inhibitors.

In some embodiments, one or more dosage forms of the invention can be used in combination with a chemotherapeutic in the treatment of cancer, such as multiple myeloma, and may improve the treatment response as compared to the response to the chemotherapeutic agent alone, without exacerbation of its toxic effects. Examples of additional pharmaceutical agents used in the treatment of multiple myeloma, for example, can include, without limitation, melphalan, melphalan plus prednisone [MP], doxorubicin, dexamethasone, and Velcade (bortezomib). Further additional agents used in the treatment of multiple myeloma include Bcr-Abl, Flt-3, RAF and FAK kinase inhibitors. Additive or synergistic effects are desirable outcomes of combining a dosage form of the present invention with an additional agent. Furthermore, resistance of multiple myeloma cells to agents such as dexamethasone may be reversible upon treatment with a dosage form of the present invention. The agents can be combined with the present compounds in a single or continuous dosage form, or the agents can be administered simultaneously or sequentially as separate dosage forms.

In some embodiments, a corticosteroid such as dexamethasone is administered to a patient in combination with at the dosage form of the invention where the dexamethasone is administered intermittently as opposed to continuously.

In some further embodiments, combinations of one or more JAK inhibitors of the invention with other therapeutic agents can be administered to a patient prior to, during, and/or after a bone marrow transplant or stem cell transplant.

In some embodiments, the additional therapeutic agent is fluocinolone acetonide (Retisert®), or rimexolone (AL-2178, Vexol, Alcon).

In some embodiments, the additional therapeutic agent is cyclosporine (Restasis®).

In some embodiments, the additional therapeutic agent is a corticosteroid. In some embodiments, the corticosteroid is triamcinolone, dexamethasone, fluocinolone, cortisone, prednisolone, or flumetholone.

In some embodiments, the additional therapeutic agent is selected from Dehydrex™ (Holles Labs), Civamide (Opko), sodium hyaluronate (Vismed, Lantibio/TRB Chemedia), cyclosporine (ST-603, Sirion Therapeutics), ARG101(T) (testosterone, Argentis), AGR1012(P) (Argentis), ecabet sodium (Senju-Ista), gefarnate (Santen), 15-(s)-hydroxyeicosatetraenoic acid (15(S)-HETE), cevilemine, doxycycline (ALTY-0501, Alacrity), minocycline, iDestrin™ (NP50301, Nascent Pharmaceuticals), cyclosporine A (Nova22007, Novagali), oxytetracycline (Duramycin, MOLI1901, Lantibio), CF101 (2S,3S,4R,5R)-3,4-dihydroxy-5-[6-[(3-iodophenyl)methylamino]purin-9-yl]-N-methyl-oxolane-2-carbamyl, Can-Fite Biopharma), voclosporin (LX212 or LX214, Lux Biosciences), ARG103 (Agentis), RX-10045 (synthetic resolvin analog, Resolvyx), DYN15 (Dyanmis Therapeutics), rivoglitazone (DE011, Daiichi Sanko), TB4 (RegeneRx), OPH-01 (Ophtalmis Monaco), PCS101 (Pericor Science), REV1-31 (Evolutec), Lacritin (Senju), rebamipide (Otsuka-Novartis), OT-551 (Othera), PAI-2 (University of Pennsylvania and Temple University), pilocarpine, tacrolimus, pimecrolimus (AMS981, Novartis), loteprednol etabonate, rituximab, diquafosol tetrasodium (INS365, Inspire), KLS-0611 (Kissei Pharmaceuticals), dehydroepiandrosterone, anakinra, efalizumab, mycophenolate sodium, etanercept (Embrel®), hydroxychloroquine, NGX267 (TorreyPines Therapeutics), actemra, gemcitabine, oxaliplatin, L-asparaginase, or thalidomide.

In some embodiments, the additional therapeutic agent is an anti-angiogenic agent, cholinergic agonist, TRP-1 receptor modulator, a calcium channel blocker, a mucin secretagogue, MUC1 stimulant, a calcineurin inhibitor, a corticosteroid, a P2Y2 receptor agonist, a muscarinic receptor agonist, an mTOR inhibitor, another JAK inhibitor, Bcr-Abl kinase inhibitor, Flt-3 kinase inhibitor, RAF kinase inhibitor, and FAK kinase inhibitor such as, for example, those described in WO 2006/056399, which is incorporated herein by reference in its entirety. In some embodiments, the additional therapeutic agent is a tetracycline derivative (e.g., minocycline or doxycline). In some embodiments, the additional therapeutic agent binds to FKBP12.

In some embodiments, the additional therapeutic agent is an alkylating agent or DNA cross-linking agent; an anti-metabolite/demethylating agent (e.g., 5-flurouracil, capecitabine or azacitidine); an anti-hormone therapy (e.g., hormone receptor antagonists, SERMs, or aromotase inhibitor); a mitotic inhibitor (e.g. vincristine or paclitaxel); an topoisomerase (I or II) inhibitor (e.g. mitoxantrone and irinotecan); an apoptotic inducers (e.g. ABT-737); a nucleic acid therapy (e.g. antisense or RNAi); nuclear receptor ligands (e.g., agonists and/or antagonists: all-trans retinoic acid or bexarotene); epigenetic targeting agents such as histone deacetylase inhibitors (e.g. vorinostat), hypomethylating agents (e.g. decitabine); regulators of protein stability such as Hsp90 inhibitors, ubiquitin and/or ubiquitin like conjugating or deconjugating molecules; or an EGFR inhibitor (erlotinib).

In some embodiments, the additional therapeutic agent(s) are demulcent eye drops (also known as “artificial tears”), which include, but are not limited to, compositions containing polyvinylalcohol, hydroxypropyl methylcellulose, glycerin, polyethylene glycol (e.g. PEG400), or carboxymethyl cellulose. Artificial tears can help in the treatment of dry eye by compensating for reduced moistening and lubricating capacity of the tear film. In some embodiments, the additional therapeutic agent is a mucolytic drug, such as N-acetyl-cysteine, which can interact with the mucoproteins and, therefore, to decrease the viscosity of the tear film.

In some embodiments, the additional therapeutic agent includes an antibiotic, antiviral, antifungal, anesthetic, anti-inflammatory agents including steroidal and non-steroidal anti-inflammatoires, and anti-allergic agents. Examples of suitable medicaments include aminoglycosides such as amikacin, gentamycin, tobramycin, streptomycin, netilmycin, and kanamycin; fluoroquinolones such as ciprofloxacin, norfloxacin, ofloxacin, trovafloxacin, lomefloxacin, levofloxacin, and enoxacin; naphthyridine; sulfonamides; polymyxin; chloramphenicol; neomycin; paramomycin; colistimethate; bacitracin; vancomycin; tetracyclines; rifampin and its derivatives (“rifampins”); cycloserine; beta-lactams; cephalosporins; amphotericins; fluconazole; flucytosine; natamycin; miconazole; ketoconazole; corticosteroids; diclofenac; flurbiprofen; ketorolac; suprofen; cromolyn; lodoxamide; levocabastin; naphazoline; antazoline; pheniramine; or azalide antibiotic.

It is further appreciated that certain features of the invention, which are, for clarity, described in the context of separate embodiments, can also be provided in combination in a single embodiment (as if the embodiments of the specification are written as multiply dependent claims).

### Example 1. Preparation of Sustained Release Formulations

Sustained release tablets were prepared with the excipients being in the amounts shown in the table below. Protocol A was used for the SR1 tablets, protocol B was used for the SR2 tablets, Protocol C was used for the SR3 tablets and the 25 mg SR tablets, and Protocol D was used for the SR4 tablets.

### Protocol A:

Step 1. Individually screen the adipic acid salt of the compound of Formula I, microcrystalline cellulose, hypromelloses (Methocel K100 LV and Methocel K4M), and lactose monohydrate.
Step 2. Transfer the screened material from Step 1 to a suitable blender and mix.
Step 3. Transfer the blend from Step 2 to a suitable granulator and mix.
Step 4. Add purified water while mixing.
Step 5. Transfer the granules from Step 4 into a suitable dryer and dry until LOD is less than 3%.
Step 6. Screen the granules from Step 5.
Step 7. Mix screened Magnesium Stearate with granules in Step 6 in a suitable blender.
Step 8. Compress the final blend in Step 7 on a suitable rotary tablet press.

### Protocol B:

Step 1. Individually screen the adipic acid salt of the compound of Formula I, microcrystalline cellulose, hypromellose and pregelatinized starch.
Step 2. Transfer the screened material from Step 1 to a suitable blender and mix.
Step 3. Transfer the blend from Step 2 to a suitable granulator and mix.
Step 4. Add purified water while mixing.
Step 5. Transfer the granules from Step 4 into a suitable dryer and dry until LOD is less than 3%.
Step 6. Screen the granules from Step 5.
Step 7. Individually screened polyox, butylated hydroxytoluene and colloidal silicone dioxide.
Step 8. Transfer the granules from Step 6 and material from Step 7 into a suitable blender and mix.
Step 9. Add screened Magnesium Stearate to the material in Step 8 and continue blending.
Step 10. Compress the final blend in Step 9 on a suitable rotary tablet press.

### Protocol C:

Step 1. Individually screen lactose monohydrate, the adipic acid salt of the compound of Formula I, microcrystalline cellulose and hypromelloses through a suitable screen.
Step 2. Transfer the screened material from Step 1 to a suitable blender and mix.
Step 3. Transfer the blend from Step 2 to a suitable granulator and mix.
Step 4. Add purified water while mixing.
Step 5. Screen wet granules through a suitable screen.
Step 6. Transfer the granules from Step 5 into a suitable dryer and dry until LOD is less than 3%.
Step 7. Mill the granules from Step 6.
Step 8. Mix screened magnesium stearate with granules in Step 7 in a suitable blender.
Step 9. Compress the final blend in Step 8 on a suitable rotary tablet press.

### Protocol D:

Step 1. Individually screen pregelatinized starch, the adipic acid salt of the compound of Formula I, hypromellose, and a portion of required microcrystalline cellulose through a suitable screen.
Step 2. Transfer the screened material from Step 1 to a suitable blender and mix.
Step 3. Transfer the blend from Step 2 to a suitable granulator and mix.
Step 4. Add purified water while mixing.
Step 5. Screen wet granules through a suitable screen.
Step 6. Transfer the granules from Step 5 into a suitable dryer and dry until LOD is less than 3%.
Step 7. Mill the granules from Step 6.
Step 8. Screen the remaining portion of microcrystalline cellulose and half of the sodium bicarbonate.
Step 9. Transfer the milled granules from Step 7 and screened materials from Step 8 into a suitable blender and mix.
Step 10. Screen the remaining portion of sodium bicarbonate and mix with blend in Step 9.
Step 11. Screen magnesium stearate and mix with blend in Step 10.
Step 12. Compress the final blend in Step 11 on a suitable rotary tablet press.

SR1: Composition of 100 mg Sustained Release Tablets

| | | Weight | Composition |
|---|---|---|---|
| Component | Function | (mg/tablet) | (wt %) |
| Adipic acid salt of the | Active | 126.42 ^{a} | 21.1 |
| compound of Formula I ^{a} | | | |
| Microcrystalline | Filler | 60.0 | 10.0 |
| Cellulose | | | |
| Hypromellose | Release Control | 60.0 | 10.0 |
| (Methocel K100LV) | | | |
| Hypromellose | Release Control | 60.0 | 10.0 |
| (Methocel K4M) | | | |
| Lactose Monohydrate | Filler | 290.58 | 48.4 |
| Magnesium Stearate ^{b} | Lubricant | 3.0 | 0.5 |
| Purified Water ^{c} | Granulating Liquid | q.s. | — |

| | | |
|---|---|---|
| Total | 600.0 | 100 |

^{a} Conversion factor for adipate salt to free base is 0.7911
^{b} Added after granulation
^{c} Removed during processing

SR2: Composition of 100 mg Sustained Release Tablets

| | | Weight | Composition |
|---|---|---|---|
| Component | Function | (mg/tablet) | (wt %) |
| Adipic acid salt of | Active | 126.4 ^{a} | 21.1 |
| the compound of | | | |
| Formula I ^{a} | | | |
| Microcrystalline | Filler | 180.0 | 30.0 |
| Cellulose | | | |
| Hypromellose | Binder | 6.0 | 1.0 |
| (Methocel K100LV) | | | |
| Polyethylene Oxide | Release Control | 180.0 | 30.0 |
| (Polyox WRS 1105) ^{b} | | | |
| Pregelatinized Starch | Filler | 101.6 | 16.9 |
| Colloidal Silicon | Glidant | 3.0 | 0.5 |
| Dioxide ^{b} | | | |
| Butylated | Antioxidant | 0.012 | 0.002 |
| Hydroxytoluene ^{b} | | | |
| Magnesium Stearate ^{b} | Lubricant | 3.0 | 0.5 |
| Purified Water ^{c} | Granulating Liquid | q.s. | — |

| | | |
|---|---|---|
| Total | 600.0 | 100.0 |

^{a} Conversion factor for adipate salt to free base is 0.7911
^{b} Added after granulation
^{c} Removed during processing

SR3 (100 mg): Composition of 100 mg Sustained Release Tablets

| | | Weight | Composition |
|---|---|---|---|
| Component | Function | (mg/tablet) | (wt %) |
| Adipic acid salt of the | Active | 126.4 ^{a} | 21.1 |
| compound of Formula I ^{a} | | | |
| Microcrystalline | Filler | 108.0 | 18.0 |
| Cellulose | | | |
| Hypromellose | Release Control | 42.0 | 7.0 |
| (Methocel K100LV) | | | |
| Hypromellose | Release Control | 30.0 | 5.0 |
| (Methocel K4M) | | | |
| Lactose Monohydrate | Filler | 290.6 | 48.4 |
| Magnesium Stearate ^{b} | Lubricant | 3.0 | 0.5 |
| Purified Water ^{c} | Granulating Liquid | q.s. | — |

| | | |
|---|---|---|
| Total | 600.0 | 100.0 |

^{a} Conversion factor for adipate salt to free base is 0.7911
^{b} Added after granulation
^{c} Removed during processing

SR4: Composition of 100 mg Sustained Release Tablets

| | | Weight | Composition |
|---|---|---|---|
| Excipient | Function | (mg/tablet) | (wt %) |
| Adipic acid salt | Active | 126.4 ^{a} | 21.1 |
| of the compound of | | | |
| Formula I ^{a} | | | |
| Microcrystalline | Filler | 104.6 | 17.4 |
| Cellulose ^{d} | | | |
| Hypromellose | Release Control | 210.0 | 35.0 |
| (Methocel K100LV) | | | |
| Pregelatinized Starch | Filler | 60.0 | 10.0 |
| Sodium Bicarbonate ^{b} | Gastric Floating Aid | 96.0 | 16.0 |
| Magnesium Stearate ^{b} | Lubricant | 3.0 | 0.5 |
| Purified Water ^{c} | Granulation Liquid | q.s. | — |

| | | |
|---|---|---|
| Total | 600.0 | 100.0 |

^{a} Conversion factor for adipate salt to free base is 0.7911
^{b} Added after granulation
^{c} Removed during processing
^{d} Partial added before and partial added after granulation

25 mg SR: Composition of 25 mg Sustained Release Tablets

| | | Weight | Composition |
|---|---|---|---|
| Component | Function | (mg/tablet) | (wt %) |
| Adipic acid salt of the | Active | 31.6^{a} | 12.6 |
| compound of Formula I^{a} | | | |
| Microcrystalline | Filler | 105.0 | 42.0 |
| Cellulose | | | |
| Hypromellose, | Release Control | 25.0 | 10.0 |
| (Methocel K100LV) | | | |
| Hypromellose, | Release Control | 25.0 | 10.0 |
| (Methocel K4M) | | | |
| Lactose Monohydrate | Filler | 62.15 | 24.9 |
| Magnesium Stearate^{b} | Lubricant | 1.25 | 0.5 |
| Purified Water^{c} | Granulating Liquid | q.s. | — |

| | | |
|---|---|---|
| Total | 250 | 100.0 |

^{a}Conversion factor for adipate salt to free base is 0.7911
^{b}Added after granulation
^{c}Removed during processing

### Example 2. Preparation of the IR Formulation of the Compound of Formula I

The IR formulation used in the studies in Example 3 was prepared as 50 mg capsules with the composition shown in the table below according to Protocol E below.

### Protocol E:

Step 1. Pre-mix the required amount of the adipic acid salt of the compound of Formula I and an approximately equal amount of silicified microcrystalline cellulose (SMCC).
Step 2. Pass the mixture in Step 1 through a suitable screen (for example 40 mesh).
Step 3. Screen the remaining SMCC through the same screen used in Step 2.
Step 4. Blend the screened SMCC from Step 3 along with mixture from Step 2 in a suitable blender (for example Turbula blender) for approximately 5 minutes.
Step 5. Fill the blend into capsules to desired fill weight.

| | WEIGHT | QUANTITY |
|---|---|---|
| | COMPOSITION | PER UNIT |
| INGREDIENT | (%) | (mg) |
| Adipic acid salt of the compound | 35.11 | 63.20* |
| of Formula I | | |
| Silicified Microcrystalline Cellulose, | 64.89 | 116.80 |
| NF (Prosolv SMCC HD 90) | | |
| TOTAL | 100.00% | 180.00 |
| #2 Capsules, Hard Gelatin, White | NA | 1 each |
| Opaque | | |

*Adipic acid salt of the compound of Formula I with salt conversion factor of 0.7911

### Example 3. Relative Bioavailability Study of Sustained Release Dosage Forms

A total of 72 healthy adult subjects were enrolled in 6 cohorts (12 subjects per cohort) and randomized to treatment sequences within each cohort according to a randomization schedule. All treatments were single-dose administrations of the compound of Formula I. There was a washout period of 7 days between the treatment periods.

The SR1, SR2, SR3, and SR4 formulations were evaluated in Cohort 1, Cohort 2, Cohort 3, and Cohort 4, respectively (see Example 1 for SR1, SR2, SR3, SR4, and 25 mg SR tablets used in study). The subjects received the IR and SR treatments according to a 3-way crossover design:
Treatment A: 300 mg (6×50 mg capsule) IR formulation of the compound of Formula I administered orally after an overnight fast of at least 10 hours.
Treatment B: 300 mg (3×100 mg tablets) SR formulation of the compound of Formula I administered orally after an overnight fast of at least 10 hours.
Treatment C: 300 mg (3×100 mg tablets) SR formulation of the compound of Formula I administered orally after a high-fat meal.

The subjects in Cohort 5 received the following treatments in a 2-way crossover design:
Treatment A: 300 mg (3×100 mg tablets of the compound of Formula I) SR3 administered orally after an overnight fast of at least 10 hours.
Treatment B: 300 mg (3×100 mg tablets of the compound of Formula I) SR3 administered orally after a medium-fat meal.

The subjects in Cohort 6 received the following treatments in a 3-way crossover design:
Treatment A: 50 mg (2×25 mg tablets of the compound of Formula I (25 mg SR tablets from Example 1)) administered orally after an overnight fast of at least 10 hours.
Treatment B: 50 mg (2×25 mg tablets of the compound of Formula I (25 mg SR tablets from Example 1)) administered orally after a high-fat meal.
Treatment C: 100 mg (1×100 mg tablets) SR3 administered orally after an overnight fast of at least 10 hours.

Blood samples for determination of plasma concentrations of the compound of Formula I were collected using lavender top (K2EDTA) Vacutainer® tubes at 0, 0.25, 0.5, 1, 1.5, 2, 3, 4, 6, 8, 12, 16, 24, 36, and 48 hours post dose.

Plasma samples were assayed by a validated, GLP, LC/MS/MS method with a linear range of 5.0 to 5000 nM. Table 1 summarizes the accuracy and precision (CV %) of the assay quality control samples during the analysis of the plasma samples from this study.

TABLE 1
Accuracy and Precision of the Plasma Assay Quality Control Samples

| Analyte | Low QC | | | Middle QC | | | High QC | | |
|---|---|---|---|---|---|---|---|---|---|
| (Unit) | Theo | Accuracy | CV % | Theo | Accuracy | CV % | Theo | Accuracy | CV % |
| Compound | 15.0 | 99.0% | 4.6% | 250 | 101% | 4.2% | 4000 | 99.5% | 2.2% |
| of Formula I | | | | | | | | | |

CV % = percent coefficient of variability;
QC = quality control;
Theo = theoretical or nominal concentration.

For the PK analysis, the actual sample collection times were used. For any sample with missing actual collection time, the scheduled time was used provided that there was no protocol deviation noted for the collection of these samples.

Standard noncompartmental PK methods were used to analyze the data for the plasma concentration of the compound of Formula using Phoenix WinNonlin version 6.0 (Pharsight Corporation, Mountain View, CA). Thus, Cₘₐₓ and Tₘₐₓ were taken directly from the observed plasma concentration data. The terminal-phase disposition rate constant (λ_{z}) was estimated using a log-linear regression of the concentration data in the terminal disposition phase, and t_{1/2} was estimated as 1n(2)/λ_{z}. AUC₀₋ₜ was estimated using the linear trapezoidal rule for increasing concentrations and the log-trapezoidal rule for decreasing concentrations, and the total AUC_{0-∞}. was calculated as AUC₀₋ₜ+Cₜ/λ_{z}. The oral-dose clearance (CL/F) was estimated as Dose/AUC_{0-∞}, and the terminal-phase volume of distribution (V_{z}/F) was estimated as Dose/[AUC_{0-∞}*80 _{z}].

The log-transformed Cₘₐₓ and AUC values (after dose normalization, where the doses were different) were compared between the fasted and fed dosing treatments, and between the SR and IR dosing treatments, using a crossover ANOVA (fixed factor=treatment, sequence and period, random effect=subject (sequence)). The adjusted geometric mean ratios of Cₘₐₓ and AUC between the treatments (reference=IR or fasted administration of SR) and the corresponding 90% confidence intervals (CIs) were determined. In addition, the correlation between the observed food effect of a high-fat meal on AUC_{0-∞}and the relative bioavailability of the SR formulations (with reference to the IR capsule) were explored by a quantile plot using the data from all subjects who completed Treatment A, B, and C in Cohorts 1 to 4. The statistical analysis was performed using Phoenix WinNonlin version 6.0.

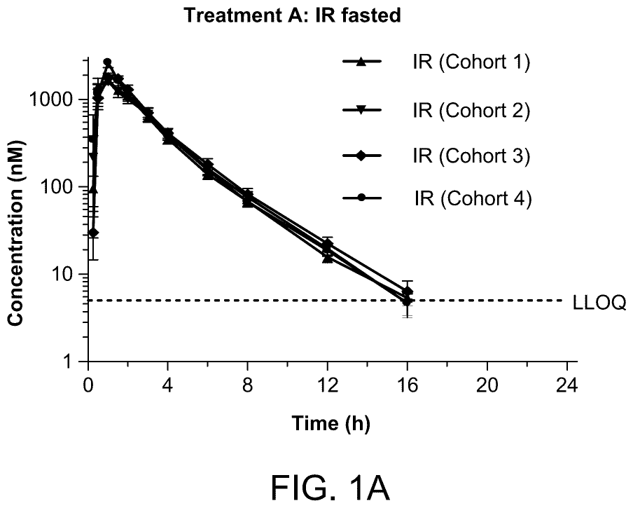

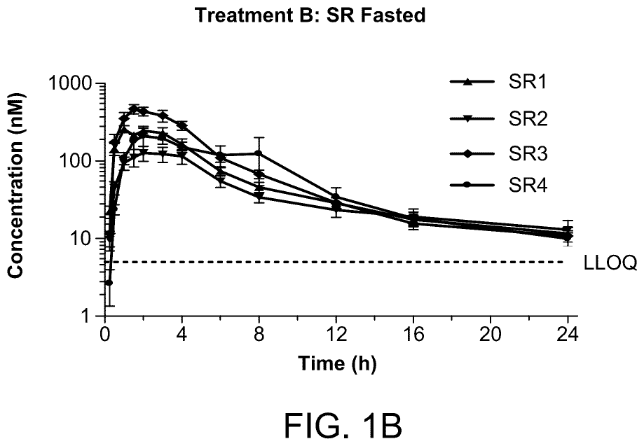

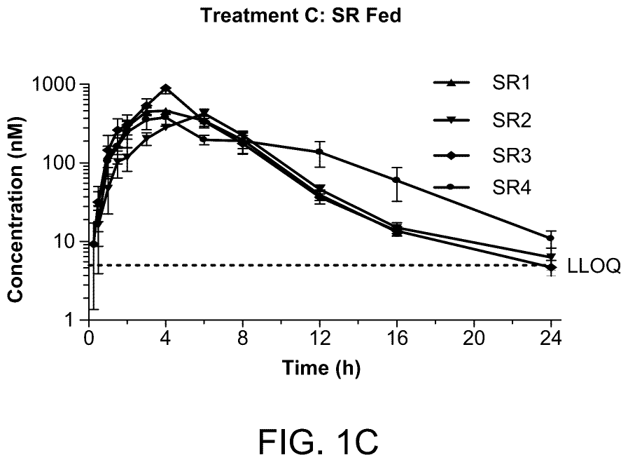

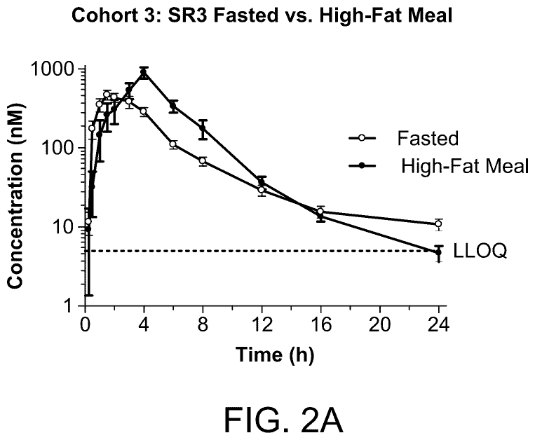

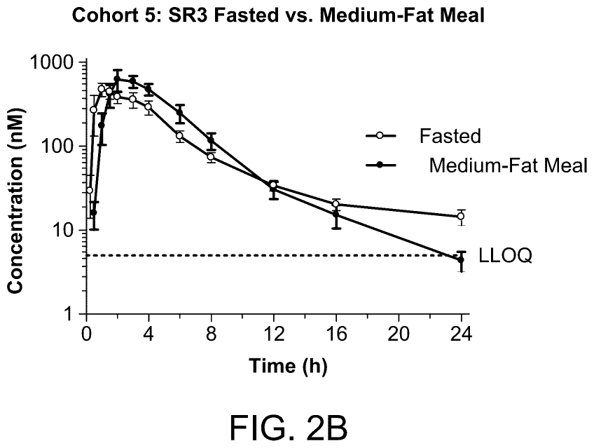

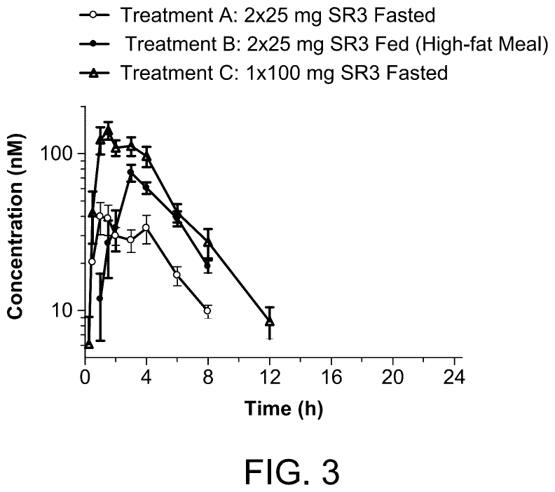

FIG. 1 presents plasma concentrations of the compound of Formula I (mean±SE) for the subjects in Cohorts 1 to 4 following Treatment A (300 mg IR administration in fasted state), Treatment B (300 mg SR administration in fasted state), and Treatment C (300 mg SR administration with a high-fat meal). FIG. 2 compares the effect of a high-fat meal and medium-fat meal on the mean PK profile following a single-dose 300 mg (3×100 mg) administration of the compound of Formula I SR3 tablets. FIG. 3 presents plasma concentrations of the compound of Formula I (mean±SE) for the subjects in Cohort 6 following Treatment A (2×25 mg SR tablet administration in fasted state), Treatment B (2×25 mg SR tablet with a high-fat meal), and Treatment C (1×100 mg SR3 administration in fasted state).

Tables 2A, 2B, 3A and 3B summarize mean PK parameters for subjects in Cohorts 1 to 4, the relative bioavailability (reference=IR capsule) and food effect (high-fat meal) for the 100 mg strength SR1-SR4 tablets. Table 4A and 4B summarize mean PK parameters for subjects in Cohort 5, and food effect (medium-fat meal) for the 100 mg strength SR3 tablet. Table 5A and 5B summarizes mean PK parameters for subjects in Cohort 6, the dose-normalized relative bioavailability (reference=100 mg SR3 tablet), and the food effect (high-fat meal) for the 25 mg SR tablet.

TABLE 2A

| Cohort/ | | Cₘₐₓ | Tₘₐₓ | Cₘₐₓ/ | t_{1/2} |
|---|---|---|---|---|---|
| Treatment | n | (μM) | (h) | C_{12 h} | (h) |
| Cohort 1 | | | | | |
| 300 mg IR | 12 | 2.29 ± 0.50 | 1.0 | 197 ± 147 | 2.0 ± 0.27 |
| (fasted) | | 2.24 | (0.50-2.0) | 159 | 2.0 |
| 300 mg SR1 | 12 | 0.341 ± 0.13 | 1.3 | 13.2 ± 7.8 | 9.2 ± 4.5 |
| (fasted) | | 0.317 | (0.50-3.0) | 11.6 | 8.3 |
| 300 mg SR1 | 12 | 0.610 ± 0.14 | 4.0 | 18.0 ± 6.4 | 3.2 ± 1.4 |
| (high-fat | | 0.595 | (2.0-8.0) | 16.8 | 3.0 |
| meal) | | | | | |
| Cohort 2 | | | | | |
| 300 mg IR | 12 | 2.05 ± 0.67 | 1.0 | 130 ± 72.9 | 2.1 ± 0.34 |
| (fasted) | | 1.92 | (0.50-3.0) | 112 | 2.1 |
| 300 mg SR2 | 12 | 0.191 ± 0.10 | 2.5 | 11.4 ± 9.9 | 11 ± 8.4 |
| (fasted) | | 0.172 | (1.0-4.0) | 8.60 | 9.23 |
| 300 mg SR2 | 12 | 0.470 ± 0.16 | 6.0 | 11.0 ± 4.0 | 3.5 ± 2.6 |
| (high-fat | | 0.443 | (1.5-6.0) | 10.4 | 3.0 |
| meal) | | | | | |
| Cohort 3 | | | | | |
| 300 mg IR | 11 | 2.35 ± 0.41 | 1.0 | 136 ± 70.8 | 2.2 ± 0.53 |
| (fasted) | | 2.31 | (0.50-2.0) | 120 | 2.2 |
| 300 mg SR3 | 11 | 0.553 ± 0.24 | 1.5 | 22.9 ± 13.4 | 9.8 ± 8.5 |
| (fasted) | | 0.502 | (0.50-3.0) | 19.3 | 7.2 |
| 300 mg SR3 | 12 | 1.05 ± 0.47 | 4.0 | 34.9 ± 15.8 | 3.3 ± 1.2 |
| (high-fat | | 0.968 | (1.5-8.0) | 30.8 | 3.1 |
| meal) | | | | | |
| Cohort 4 | | | | | |
| 300 mg IR | 12 | 2.94 ± 0.98 | 1.0 | 170 ± 58.6 | 2.1 ± 0.58 |
| (fasted) | | 2.78 | (0.25-1.5) | 162 | 2.1 |
| 300 mg SR4 | 12 | 0.321 ± 0.27 | 2.0 | 10.3 ± 6.0 | 7.3 ± 5.3 |
| (fasted) | | 0.249 | (1.5-8.1) | 8.92 | 6.0 |
| 300 mg SR4 | 12 | 0.549 ± 0.28 | 4.0 | 12.8 ± 14.8 | 4.9 ± 2.6 |
| (high-fat | | 0.481 | (2.0-16) | 6.06 | 4.4 |
| meal) | | | | | |

TABLE 2B

| Cohort/ | AUC₀₋ₜ | AUC_{0-∞} | CL/F |
|---|---|---|---|
| Treatment | (μM * h) | (μM * h) | (L/h) |
| Cohort 1 | | | |
| 300 mg IR | 4.43 ± 1.00 | 4.45 ± 1.00 | 127 ± 27.1 |
| (fasted) | 4.33 | 4.35 | 124 |
| 300 mg SR1 | 1.55 ± 0.54 | 1.65 ± 0.54 | 359 ± 106 |
| (fasted) | 1.47 | 1.57 | 345 |
| 300 mg SR1 | 2.88 ± 0.65 | 2.91 ± 0.65 | 194 ± 39.9 |
| (high-fat meal) | 2.82 | 2.85 | 190 |
| Cohort 2 | | | |
| 300 mg IR | 4.45 ± 1.36 | 4.47 ± 1.36 | 134 ± 50.1 |
| (fasted) | 4.24 | 4.27 | 127 |
| 300 mg SR2 | 1.00 ± 037 | 1.17 ± 0.43 | 510 ± 148 |
| (fasted) | 0.95 | 1.11 | 488 |
| 300 mg SR2 | 2.48 ± 0.70 | 2.52 ± 0.72 | 235 ± 83.5 |
| (high-fat meal) | 2.38 | 2.42 | 224 |
| Cohort 3 | | | |
| 300 mg IR | 5.00 ± 1.33 | 5.03 ± 1.34 | 115 ± 32.4 |
| (fasted) | 4.83 | 4.87 | 111 |
| 300 mg SR3 | 2.28 ± 0.71 | 2.39 ± 0.70 | 248 ± 82.8 |
| (fasted) | 2.17 | 2.29 | 236 |
| 300 mg SR3 | 3.55 ± 1.13 | 3.59 ± 1.13 | 165 ± 50.2 |
| (high-fat meal) | 3.40 | 3.44 | 158 |
| Cohort 4 | | | |
| 300 mg IR | 5.23 ± 2.16 | 5.25 ± 2.15 | 117 ± 39.8 |
| (fasted) | 4.88 | 4.90 | 111 |
| 300 mg SR4 | 1.61 ± 1.23 | 1.70 ± 1.25 | 456 ± 259 |
| (fasted) | 1.31 | 1.40 | 387 |
| 300 mg SR4 | 3.00 ± 1.17 | 3.13 ± 1.20 | 200 ± 80.0 |
| (high-fat meal) | 2.78 | 2.92 | 186 |

TABLE 3A

| | | | | |
|---|---|---|---|---|
| Cohort/ | Cₘₐₓ | Tₘₐₓ | Cₘₐₓ/ | t_{1/2} |
| Treatment | (μM) | (h) | C_{12 h} | (h) |

| | |
|---|---|
| SR1 fasted vs IR | 14.2% |
| | (11.4%-17.5%) |
| SR1 fed vs fasted | 188% |
| | (152%-232%) |
| SR2 fasted vs IR | 8.9% |
| | (6.7%-11.9%) |
| SR2 fed vs fasted | 258% |
| | (193%-344%) |
| SR3 fasted vs IR | 22.3% |
| | (17.4%-28.6%) |
| SR3 fed vs fasted | 191% |
| | (150%-244%) |
| SR4 fasted vs IR | 9.0% |
| | (6.8%-11.9%) |
| SR4 fed vs fasted | 193% |
| | (146%-256%) |

PK parameter values are mean ± SD and geometric mean except for Tₘₐₓ, where median (90% confidence interval) is reported.

TABLE 3B

| | | | |
|---|---|---|---|
| Cohort/ | AUC₀₋ₜ | AUC_{0-∞} | CL/F |
| Treatment | (μM * h) | (μM * h) | (L/h) |

Geometric Mean Relative Bioavailability
and the 90% Confidence Intervals

| | | |
|---|---|---|
| SR1 fasted vs IR | 34.1% | 36.1% |
| | (31.3%-37.0%) | (33.3%-39.2%) |
| SR1 fed vs fasted | 191% | 181% |
| | (176%-208%) | (167%-196%) |
| SR2 fasted vs IR | 22.4% | 26.0% |
| | (18.3%-27.4%) | (21.6%-31.3%) |
| SR2 fed vs fasted | 250% | 218% |
| | (204%-306%) | (181%-262%) |
| SR3 fasted vs IR | 45.4% | 47.5% |
| | (39.6%-52.0%) | (41.9%-53.9%) |
| SR3 fed vs fasted | 151% | 145% |
| | (132%-173%) | (128%-164%) |
| SR4 fasted vs IR | 26.9% | 28.5% |
| | (21.6%-33.4%) | (23.2%-35.1%) |
| SR4 fed vs fasted | 213% | 215% |
| | (171%-264%) | (172%-268%) |

PK parameter values are mean ± SD and geometric mean except for Tₘₐₓ, where median (90% confidence interval) is reported.

TABLE 4A

| Cohort/ | | Cₘₐₓ | Tₘₐₓ | Cₘₐₓ/ | t_{1/2} |
|---|---|---|---|---|---|
| Treatment | n | (μM) | (h) | C_{12 h} | (h) |
| Cohort 5 | | | | | |
| 300 mg | 12 | 0.619 ± 0.41 | 1.75 | 22.8 ± 16.7 | 7.7 ± 5.2 |
| SR3 | | 0.523 | (0.50-4.0) | 17.8 | 6.2 |
| (fasted) | | | | | |
| 300 mg | 12 | 0.875 ± 0.47 | 2.5 | 40.6 ± 22.7 | 3.6 ± 2.0 |
| SR3 | | 0.764 | (1.5-6.0) | 31.2 | 3.3 |
| (medium- | | | | | |
| fat meal) | | | | | |

Geometric Mean Relative Bioavailability
and the 90% Confidence Intervals

| | |
|---|---|
| SR3 | 146% |
| fed vs | (105%-202%) |
| fasted | |

Pharmacokinetic parameter values are mean ± SD and geometric mean except for Tₘₐₓ, where median (90% confidence interval) is reported.

TABLE 4B

| Cohort/ | AUC₀₋ₜ | AUC_{0-∞} | CL/F |
|---|---|---|---|
| Treatment | (μM * h) | (μM * h) | (L/h) |
| Cohort 5 | | | |
| 300 mg SR3 | 2.46 ± 1.13 | 2.58 ± 1.12 | 251 ± 105 |
| (fasted) | 2.23 | 2.36 | 230 |
| 300 mg SR3 | 2.98 ± 1.34 | 3.02 ± 1.35 | 215 ± 94.2 |
| (medium-fat | 2.72 | 2.76 | 196 |
| meal) | | | |

Geometric Mean Relative Bioavailability
and the 90% Confidence Intervals

| | | |
|---|---|---|
| SR3 fed vs | 122% | 117% |
| fasted | (102%-146%) | (99.9%-137%) |

Pharmacokinetic parameter values are mean ± SD and geometric mean except for Tₘₐₓ, where median (90% confidence interval) is reported.

TABLE 5A

| Cohort/ | | Cₘₐₓ | Tₘₐₓ | Cₘₐₓ/ | t_{1/2} |
|---|---|---|---|---|---|
| Treatment | n | (nM) | (h) | C_{12 h} | (h) |
| Cohort 6 | | | | | |
| 2 × 25 mg SR3 | 12 | 55.1 ± 30.3 | 1.3 | NR | 4.0 ± 2.6 |
| (fasted) | | 48.0 | (0.50-4.0) | | 3.4 |
| 2 × 25 mg SR3 | 12 | 80.3 ± 27.3 | 3.0 | NR | 2.2 ± 0.4 |
| (high-fat meal) | | 76.7 | (1.5-6.0) | | 2.2 |
| 1 × 100 mg SR3 | 11 | 174 ± 69.5 | 1.8 | NR | 3.0 ± 1.3 |
| (fasted) | | 161 | (0.50-4.0) | | 2.7 |

Geometric Mean Relative Bioavailability
and the 90% Confidence Confidence Intervals

| | |
|---|---|
| 2 × 25 mg SR3 | 160% |
| fed vs fasted | (129%-199%) |
| 2 × 25 mg SR3 | 58.7%ⁱ⁾ |
| vs 1 × 100 mg SR3 | (46.9%-73.5%) |
| (fasted) | |

NC = not calculated because of significant numbers of mismatching Tₗₐₛₜ within the subjects between treatments; NR = not reported because significant numbers of C_{12 h} values were BQL.
PK parameter values are mean ± SD and geometric mean except for Tₘₐₓ, where median (90% confidence interval) is reported.
ⁱ⁾Statistical comparison was dose-normalized.

TABLE 5B

| Cohort/ | AUC₀₋ₜ | AUC_{0-∞} | CL/F |
|---|---|---|---|
| Treatment | (nM * h) | (nM * h) | (L/h) |
| Cohort 6 | | | |
| 2 × 25 mg SR3 | 205 ± 103 | 243 ± 99.9 | 429 ± 167 |
| (fasted) | 183 | 226 | 400 |
| 2 × 25 mg SR3 | 333 ± 104 | 376 ± 94.6 | 253 ± 57.7 |
| (high-fat meal) | 319 | 366 | 247 |
| 1 × 100 mg SR3 | 671 ± 230 | 704 ± 230 | 280 ± 81.5 |
| (fasted) | 639 | 673 | 268 |

Geometric Mean Relative Bioavailability
and the 90% Confidence Intervals

| | | |
|---|---|---|
| 2 × 25 mg SR3 | 174% | 158% |
| fed vs fasted | (150%-202%) | (138%-182%) |
| 2 × 25 mg SR3 | NC | 66.1%ⁱ⁾ |
| vs 1 × 100 mg SR3 | | (57.5%-75.9% |
| (fasted) | | |

NC = not calculated because of significant numbers of mismatching Tₗₐₛₜ within the subjects between treatments; NR = not reported because significant numbers of C_{12 h} values were BQL.
PK parameter values are mean ± SD and geometric mean except for Tₘₐₓ, where median (90% confidence interval) is reported.
ⁱ⁾Statistical comparison was dose-normalized.

The mean PK profiles following the fasting single-dose administration of 300 mg IR capsules were similar among the subjects in Cohorts 1 to 4 (FIG. 1). Compared to the IR formulation, following fasting single-dose administration of the SR1-SR4 formulations (3×100 mg tablets), the observed plasma median Tₘₐₓ values were moderately prolonged (by 0.3 to 1.5 hours) with significantly reduced mean Cₘₐₓ values (the upper bounds of the 90% CI for the geometric mean Cₘₐₓ ratios were <30%), suggesting decreased absorption rate of the compound of Formula I for the SR tablets. The apparent mean disposition t_{1/2} observed in the terminal phase was significantly longer, ranging from 7.3 to 11 hours for SR1-SR4, as compared to about 2 hours for the IR capsule, indicating that the systemic elimination of the compound of Formula I was likely rate-limited by its absorption, which was sustained in the terminal disposition phase. As a result of lower Cₘₐₓ and longer disposition t_{1/2}, the Cₘₐₓ/C₁₂ₕ ratios were significantly lower for the SR tablets compared to the IR capsule for the same subjects studied. The geometric mean Cₘₐₓ/C₁₂ₕ ratios were 11.6-, 8.6-, 19.3-, and 8.9-fold, respectively, for SR1, SR2, SR3, and SR4 tablets, as compared to 112- to 162-fold for the IR capsules administered in the fasted state.

For administration in the fasted state, the 4 SR tablets showed reduced relative bioavailability compared to the IR capsule dosed in the same subjects. The percent geometric mean ratios (90% CI) of Cₘₐₓ were 14.2% (11.4%-17.5%), 8.9% (6.7%-11.9%), 22.3% (17.4%-28.6%) and 9.0% (6.8%-11.9%) for SR1, SR2, SR3, and SR4, respectively. The percent geometric mean ratios (90% CI) of AUC_{0-∞} were 36.1% (33.3%-39.2%), 26.0% (21.6%-31.3%), 47.5% (41.9%-53.9%), and 28.5% (23.2%-35.1%) for SR1, SR2, SR3, and SR4, respectively. SR3 and SR1 demonstrated the best and second best relative bioavailability, respectively, among the SR formulations tested.

Dosed in the fasted state, the intersubject variability as measured by percent coefficient of variability (CV %) in plasma exposure was significantly higher for the gastroretentive formulation SR4, but comparable among the 3 regular SR tablets designed for intestinal release. The intersubject CV % for the 100 mg SR1 tablet was 39% and 33% for Cₘₐₓ and AUC_{0-∞}, respectively. The intersubject CV % for the 100 mg SR2 tablet was 50% and 37% for Cₘₐₓ and AUC_{0-∞}, respectively. The intersubject CV % for the 100 mg SR3 tablet was 43% and 29% for Cₘₐₓ and AUC_{0-∞}, respectively. The intersubject CV % for the 100 mg SR4 tablet was 83% and 73% for Cₘₐₓ and AUC_{0-∞}, respectively. Pooling all subjects in Cohorts 1-5 (n=59) who were administered 300 mg IR in the fasted state, the intersubject CV % was 49% and 39% for Cₘₐₓ and AUC_{0-∞}, respectively, comparable to the CV % values observed for SR1, SR2, and SR3.

A positive food effect was observed for all SR formulations studied at the 300 mg (3×100 mg) dose level. Administered after a high-fat meal, geometric mean Cₘₐₓ and AUC_{0-∞} values increased by 88% and 81%, respectively, for SR1; by 158% and 118%, respectively; for SR2; by 91% and 45%; respectively; for SR3; and by 93% and 115%; respectively; for SR4. The food effect was moderate for a medium-fat meal as compared to a high-fat meal, as suggested by the data for SR3 in Cohort 5. For SR3, Cₘₐₓ and AUC_{0-∞} values increased by 46% and 17%, respectively, when it was administered following a standardized medium-fat meal. Administration with food did not significantly change the intersubject CV % in compound of Formula I plasma exposure for SR1, SR2, and SR3, which are SR formulations designed for intra-intestinal release. For SR4, which is a gastroretentive SR formulation, the intersubject CV % in plasma exposures appeared to be significantly reduced with a concomitant high-fat meal.

This study also explored the dose-normalized relative bioavailability of the 25 mg SR tablet in reference to the 100 mg SR3 tablet. For the subjects in Cohort 6, the dose-normalized Cₘₐₓ and AUC_{0-∞} percent geometric mean ratio for the 2×25 mg SR3treatment was 59% and 66%, respectively, versus the 1×100 mg SR3 administration in the fasted state. However, due to the supralinear dose-exposure relationship for the compound of Formula I, the relative bioavailability of the 25 mg SR tablet may be underestimated. For the 2×25 mg SR dose, a high-fat meal increased compound of Formula I Cₘₐₓ and AUC_{0-∞} by 60% and 58%, respectively.

For the four SR formulations evaluated, the observed apparent disposition t_{1/2}, was comparable, and the Cₘₐₓ/C₁₂ₕ ratios from a fasting single-dose administration (which is used as a proxy for P/T ratio from twice-daily administration) were similar among SR1, SR2, and SR4 (˜10-fold) and moderately higher for SR3 (˜20-fold). Overall, all 4 SR formulations demonstrated a significantly flatter PK profile compared the IR capsule, meeting an important objective for sustained release. Bioavailability of orally administered drug products may be defined by the rate and extent of the drug absorption into systemic circulation. A reduction in drug absorption rate by limiting the drug release rate from drug products is a design requirement in sustained release formulations.

Therefore, for SR formulations, the extent of the compound of Formula I absorption as measured by the plasma AUC_{0-∞} is used as the primary endpoint to assess the relative bioavailability. Thus, the mean relative bioavailability is similar between SR2 (26%) and SR4 (29%), which was slightly lower than that of SR1 (36%). The best relative bioavailability was observed for SR3 (48%). The results are in line with the in vitro dissolution profiles obtained before conducting this study.

There was an apparent inverse correlation between the food effect and relative bioavailability for the SR formulations. On average, dosed with a high-fat meal, the food-effect measured by the increase in AUC_{0-∞} was the greatest for SR2 (118%) and SR4 (115%), which was lower than that for SR1 (81%). The smallest food effect was observed for SR3 (45%). This correlation was also apparent when the data from all the subjects were pooled together. A quantile plot using the pooled individual data (divided into 5 bins with 9 subjects per bin) suggests that the food effect was more significant (>2-fold increase in AUC) for the subjects with relative bioavailability less than 35%, regardless of the formulation. The food effect was moderate (˜50% or less increase in AUC) for the subjects with relative bioavailability greater than 40%, regardless of formulation. SR3 delivered a mean relative bioavailability of 48% and is likely to be associated with a moderate food effect. In fact, when the SR3 tablet (3×100 mg) was dosed with a medium-fat meal (which is a more typical daily diet), the observed increase in geometric mean AUC_{0-∞} was only 17%, suggesting that this formulation may be administered without regard to medium-or low-fat meals. From the perspective of avoiding significant food effect, SR3 is superior to the other formulations.

### Example 4. Clinical Results in Phase 2a in Patients With Active Rheumatoid Arthritis (RA)

An initial 28 day part of the study was conducted in order to select doses moving forward, guiding dose selection for the 3 month second part of the study. Part 2 of the study was randomized, double-blind, placebo controlled (sponsor unblinded) with treatment for 84 days. Sixty subjects to be randomized, using the same population as in Part 1: single cohort, five parallel treatment groups, 12 subjects each: 100 mg SR3 tablets BID; 300 mg (3×100 mg SR3 tablets) QD; 200 mg (2×100 mg SR3 tablets) BID; 600 mg (6×100 mg SR3 tablets) QD; and placebo. Interim data was submitted to ACR (American College of Rheumatology) 2013 (n=40 subjects who completed day 84). The ACR scores at 3 months re shown in Table 6. The ACR scores for the 600 mg QD are unprecedented as compared to other JAK inhibitors that are approved for treatment of RA. For example, the approved product for tofacitinib citrate (5 mg BID) showed much lower ACR scores at 3 months: 59% (ACR20), 31% (ACR50), and 15% (ACR70) (Table 5 of XELJANZ®-tofacitinib citrate tablet-label).

TABLE 6

| | | 100 mg | 300 mg | 200 mg | 600 mg |
|---|---|---|---|---|---|
| | Placebo | BID | QD | BID | QD |
| ACR20 | 38 | 50 | 44 | 50 | 100 |
| ACR50 | 25 | 38 | 44 | 38 | 71 |
| ACR70 | 13 | 25 | 22 | 13 | 57 |

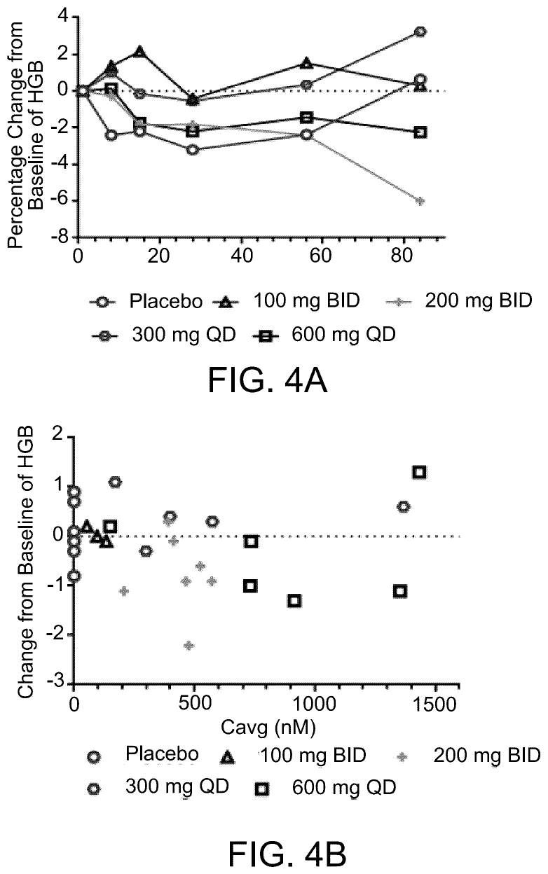

The percent change from baseline for hemoglobin was also studied for each of the dosing regimens (FIG. 4A as a function of days; FIG. 4B as a function of total average concentration (Cavg)). As can be seen in FIG. 4A-B, the 200 mg BID dose showed a drop away from the baseline compared to the other doses which tended to stay close to the placebo levels. For example, the 600 mg QD dose did not show the same downward trend as shown for the BID dose. However, as can be seen in Table 6, the once-daily dosing (600 mg QD) did not compromise efficacy compared with the BID doses. This indicates that the once-daily dosing (such as 600 mg QD) may achieve maximal efficacy without inducing side-effects such anemia. As shown in FIG. 4 and Table 6, the 600 mg QD dose has robust efficacy with trivial change in hemoglobin levels.

It is believed that this efficacy/side-effect profile may be due to the QD dose achieving maximal JAK1 signaling (tied to efficacy) with low JAK2 inhibition at the trough, as JAK2 signaling is tied to hematopoiesis. This hypothesis is supported by the PK derived JAK1 (IL-6) and JAK2 (TPO) inhibition data for the compound of Formula at various doses (Table 7). In particular, the 600 mg QD dose showed similar average IL-6 inhibition to the 200 mg BID and 400 mg BID doses (61% versus 64% and 69%), but lower trough TPO inhibition in comparison to the 200 mg BID and 400 mg BID doses (4% versus 13% and 16%). The trough IL-6 inhibition for the 600 mg QD dose is also lower than the trough IL-6 inhibition for the 200 mg BID and 400 mg BID doses, which suggests that there may be a reduction in infection from the QD dose.

TABLE 7

| | Average | Trough | Average | Trough |
|---|---|---|---|---|
| | IL-6 | IL-6 | TPO | TPO |
| Dose regimen | inhibition | inhibition | inhibition | inhibition |
| 100 mg QD | 30% | 7% | 7% | <1% |
| 200 mg QD | 39% | 11% | 11% | <1% |
| 300 mg QD | 47% | 16% | 18% | 1% |
| 600 mg QD | 61% | 31% | 36% | 4% |
| 100 mg BID | 44% | 22% | 11% | 2% |
| 200 mg BID | 64% | 52% | 24% | 13% |
| 400 mg BID | 69% | 56% | 33% | 16% |

### Example 5. Clinical Results in Patients with Plaque Psoriasis

A double-blind (sponsor unblinded), randomized, placebo controlled study was conducted in approximately 48 subjects treated for 28 days. Eligibility requirements included: active plaque psoriasis for at least 6 months at screening; body surface area (BSA) of plaque psoriasis of ≥5%; psoriasis area and severity index (PASI) score of ≥5; static physician's global assessment (sPGA) score of ≥3; inadequate response to topical therapies; innovative design allowing rapid progress between doses, with conservative safety assessment. Four staggered dose groups of 12 subjects each (9 active and 3 PBO) progressing from 100 mg QD to 200 mg QD to 200 mg BID to 600 mg QD. Once the 4th subject (block of 3 active 1 PBO) completed 28 days administration without a Grade 3 or higher AE, the next group of 12 subjects initiated treatment with the next highest dose; while the first 4 subjects in this group are treated for 28 days, the 1st group is filled 60 subjects with moderate to severe psoriasis were randomized. There were five treatment groups: placebo, 100 mg QD, 200 mg QD, 200 mg BID and 600 mg QD. A sequential method of recruitment was used, increasing from the lowest dose to the highest, each after the completion of 28 days for the first four subjects in the previous dose. The results at 28 days are show in Table 8 (PASI 50 is Psoriasis Area and Severity Index). These PASI 50 score of 81.8% for the 600 mg QD dose are unprecedented as compared to other JAK inhibitors that are in development for treatment of psoriasis. For example, 5 mg tofacitinib (also known as tasocitinib) showed lower PASI 50 score of 65.3% at 12 weeks (published on http://press.pfizer.com on Oct. 7, 2010). The 5 mg tofacitinib dose is the approved dosage level for RA for safety reasons in the US.

TABLE 8

| | | 100 mg | 200 mg | 200 mg | 600 mg |
|---|---|---|---|---|---|
| | Placebo | BID | QD | BID | QD |
| Mean % | −12.5% | −22.2% | −29.4% | −35.2% | −42.4% |
| change | | | | | |
| sPGA | | | | | |
| % sPGA | 0 | 11.1% | 22.2% | 33.3% | 45.5% |
| (clear or | | | | | |
| minimal) | | | | | |
| % PASI 50 | 8.3% | 22.2% | 66.7% | 44.4% | 81.8% |

### Example 6. Open-Label Phase II Study in Patients With Myelofibrosis

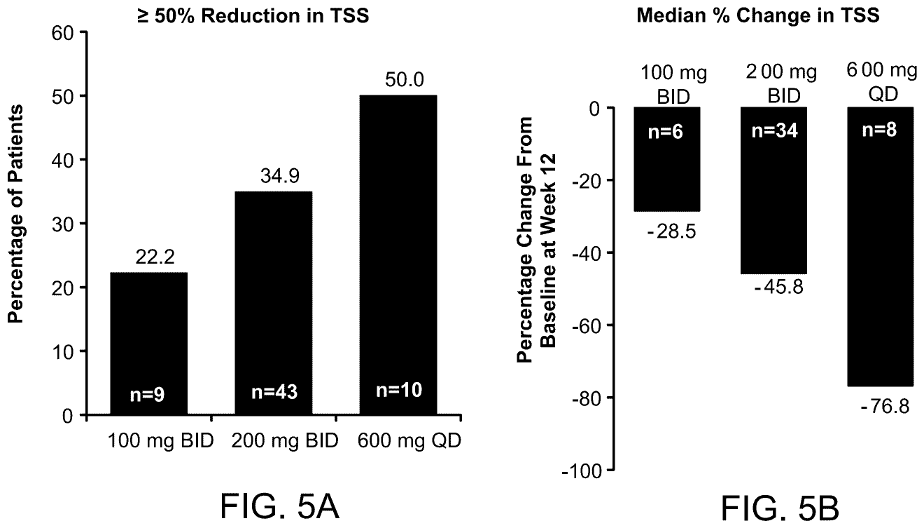

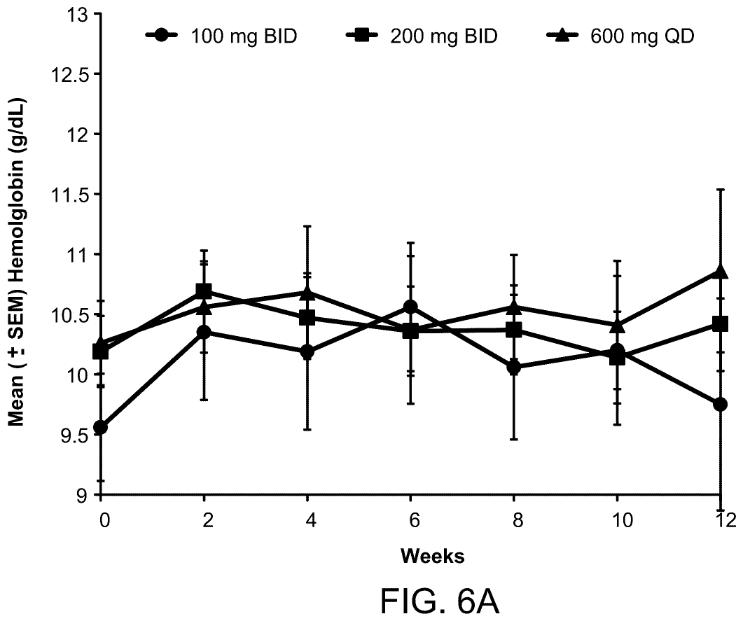

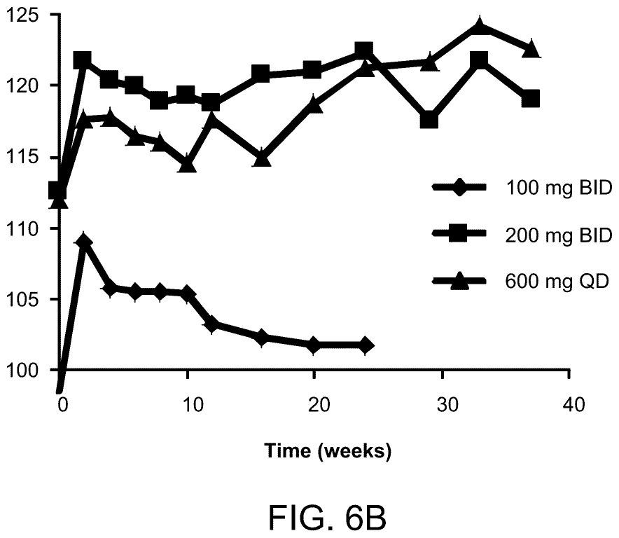

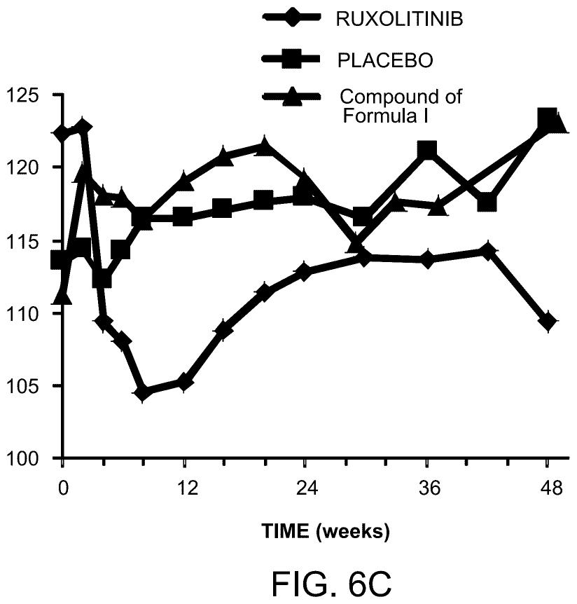

In this study, patients with age ≥18 years, a diagnosis of primary myelofibrosis (PMF) or post-polycythemia vera MF or post-essential thrombocythemia MF (JAK2V617F positive or negative mutation status), platelet counts≥50×109/L, hemoglobin levels≥8.0 g/dL (transfusions permitted to achieve these levels), intermediate-1 or higher per DIPSS criteria, and palpable spleen or prior splenectomy were enrolled. Three different dose cohorts were assessed: (1) 100 mg SR3 tablets BID) (2) 200 mg (2×100 mg SR3 tablets) BID; and (3) 600 mg (6×100 mg SR3 tablets) QD. FIG. 5A-B show interim results with respect to proportion of subjects with ≥50% reduction in total symptom score (TSS) in each dose group per the modified Myelofibrosis Symptom Assessment Form (MFSAF) v3.0 electronic diary at week 12 compared with baseline (The modified MFSAF v3.0 comprises 19 questions assessing MF-related symptoms on a scale of 0 (absent) to 10 (worst imaginable)). FIG. 5A depicts the percentage of patients having a ≥50% reduction in TSS at week 12 by dose cohort (100 mg BID, 200 mg BID, and 600 mg QD) (patients who discontinued prior to the week 12 visit were considered nonresponders). FIG. 5B depicts the percent change in TSS from baseline at week 12 by dose cohort (100 mg BID, 200 mg BID, and 600 mg QD) (only patients with baseline and week 12 data were included). FIG. 6A depicts mean hemoglobin levels (g/dL) over time by dose cohort (100 mg BID, 200 mg BID, and 600 mg QD) (interim results of study for all patients). FIG. 6B depicts mean hemoglobin levels (g/dL) over time by dose cohort (100 mg BID, 200 mg BID, and 600 mg QD) at 48 weeks. FIG. 6C depicts mean hemoglobin levels (g/dL) over time by dose cohort at 48 weeks as an average for three dose cohorts as compared to individuals dosed with placebo or ruxolitinib (ruxolitinib was dosed according to the label for Jakafi®). The data show an increase in hemoglobin levels for the 600 mg QD dose. Finally, Table 9 below show interim hematology laboratory results (new and worsening) for each dose cohort. Table 9a shows the hematology laboratory results (new and worsening) for each dose cohort after long exposure.

TABLE 9

| | N % | | |
|---|---|---|---|
| Event n | 100 mg BID | 200 mg BID | 600 mg QD |
| Days of Exposure, | 102.5 | 169.0 | 16.0 |
| median (range) | (23.0, 376.0) | (22.0, 339.0) | (1.0, 196.0) |
| Anemia, Grade 3 | 3/9 (33.3) | 12/42 (28.6) | 2/29 (6.9) |
| Thrombocytopenia | | | |
| Grade 3 | 4/9 (44.4) | 12/44 (27.3) | 1/29 (3.4) |
| Grade 4 | 0/9 (0) | 2/45 (4.4) | 0/29 (0) |

TABLE 9a

| | N % | | |
|---|---|---|---|
| | 100 mg BID | 200 mg BID | 600 mg QD |
| Event n | (N = 10) | (N = 45) | (N = 32) |
| Days of Exposure, | 102.0 | 254.0 | 192.0 |
| median (range) | (23, 519) | (22, 535) | (28, 343) |
| Anemia, Grade 3 | 3/10 (30.0) | 19/45 (42.2) | 8/32 (25.0) |
| Thrombocytopenia | | | |
| Grade 3 | 4/10 (40.0) | 13/45 (28.9) | 4/32 (12.5) |
| Grade 4 | 0/10 (0.0) | 3/45 (6.7) | 1/32 (3.1) |

### Example A: In Vitro JAK Kinase Assay

The compound of Formula I herein was tested for inhibitory activity of JAK targets according to the following in vitro assay described in Park et al., Analytical Biochemistry 1999, 269, 94-104. The catalytic domains of human JAK1 (a.a. 837-1142) and JAK2 (a.a. 828-1132) with an N-terminal His tag were expressed using baculovirus in insect cells and purified. The catalytic activity of JAK1 and JAK2 was assayed by measuring the phosphorylation of a biotinylated peptide. The phosphorylated peptide was detected by homogenous time resolved fluorescence (HTRF). IC₅₀s of compounds were measured for each kinase in the 40 microL reactions that contain the enzyme, ATP and 500 nM peptide in 50 mM Tris (pH 7.8) buffer with 100 mM NaCl, 5 mM DTT, and 0.1 mg/mL (0.01%) BSA. For the 1 mM IC₅₀ measurements, ATP concentration in the reactions was 1 mM. Reactions were carried out at room temperature for 1 hr and then stopped with 20 μL 45 mM EDTA, 300 nM SA-APC, 6 nM Eu-Py20 in assay buffer (Perkin Elmer, Boston, MA). Binding to the Europium labeled antibody took place for 40 minutes and HTRF signal was measured on a Fusion plate reader (Perkin Elmer, Boston, MA). The compound of Formula I and the adipic acid salt had an IC₅₀ at JAK1 of ≤5 nM (measured at 1 mM ATP) with a JAK2/JAK1 ratio of >10 (measured at 1 mM ATP).

### Example B: Cellular Assays

Cancer cell lines dependent on cytokines and hence JAK/STAT signal transduction, for growth, can be plated at 6000 cells per well (96 well plate format) in RPMI 1640, 10% FBS, and 1 nG/mL of appropriate cytokine. Compounds can be added to the cells in DMSO/media (final concentration 0.2% DMSO) and incubated for 72 hours at 37° C., 5% CO₂. The effect of compound on cell viability is assessed using the CellTiter-Glo Luminescent Cell Viability Assay (Promega) followed by TopCount (Perkin Elmer, Boston, MA) quantitation. Potential off-target effects of compounds are measured in parallel using a non-JAK driven cell line with the same assay readout. All experiments are typically performed in duplicate.

The above cell lines can also be used to examine the effects of compounds on phosphorylation of JAK kinases or potential downstream substrates such as STAT proteins, Akt, Shp2, or Erk. These experiments can be performed following an overnight cytokine starvation, followed by a brief preincubation with compound (2 hours or less) and cytokine stimulation of approximately 1 hour or less. Proteins are then extracted from cells and analyzed by techniques familiar to those schooled in the art including Western blotting or ELISAs using antibodies that can differentiate between phosphorylated and total protein. These experiments can utilize normal or cancer cells to investigate the activity of compounds on tumor cell survival biology or on mediators of inflammatory disease. For example, with regards to the latter, cytokines such as IL-6, IL-12, IL-23, or IFN can be used to stimulate JAK activation resulting in phosphorylation of STAT protein(s) and potentially in transcriptional profiles (assessed by array or qPCR technology) or production and/or secretion of proteins, such as IL-17. The ability of compounds to inhibit these cytokine mediated effects can be measured using techniques common to those schooled in the art.

Compounds herein can also be tested in cellular models designed to evaluate their potency and activity against mutant JAKs, for example, the JAK2V617F mutation found in myeloid proliferative disorders. These experiments often utilize cytokine dependent cells of hematological lineage (e.g. BaF/3) into which the wild-type or mutant JAK kinases are ectopically expressed (James, C., et al. Nature 434:1144-1148; Staerk, J., et al. JBC 280:41893-41899). Endpoints include the effects of compounds on cell survival, proliferation, and phosphorylated JAK, STAT, Akt, or Erk proteins.

Certain compounds herein can be evaluated for their activity inhibiting T-cell proliferation. Such as assay can be considered a second cytokine (i.e. JAK) driven proliferation assay and also a simplistic assay of immune suppression or inhibition of immune activation. The following is a brief outline of how such experiments can be performed. Peripheral blood mononuclear cells (PBMCs) are prepared from human whole blood samples using Ficoll Hypaque separation method and T-cells (fraction 2000) can be obtained from PBMCs by elutriation. Freshly isolated human T-cells can be maintained in culture medium (RPMI 1640 supplemented with 10% fetal bovine serum, 100 U/ml penicillin, 100 μg/ml streptomycin) at a density of 2×106 cells/ml at 37° C. for up to 2 days. For IL-2 stimulated cell proliferation analysis, T-cells are first treated with Phytohemagglutinin (PHA) at a final concentration of 10 μg/mL for 72 h. After washing once with PBS, 6000 cells/well are plated in 96-well plates and treated with compounds at different concentrations in the culture medium in the presence of 100 U/mL human IL-2 (ProSpec-Tany TechnoGene; Rehovot, Israel). The plates are incubated at 37° C. for 72 h and the proliferation index is assessed using CellTiter-Glo Luminescent reagents following the manufactory suggested protocol (Promega; Madison, WI).

### Example C: In Vivo Anti-Tumor Efficacy

Compounds herein can be evaluated in human tumor xenograft models in immune compromised mice. For example, a tumorigenic variant of the INA-6 plasmacytoma cell line can be used to inoculate SCID mice subcutaneously (Burger, R., et al. Hematol J. 2:42-53, 2001). Tumor bearing animals can then be randomized into drug or vehicle treatment groups and different doses of compounds can be administered by any number of the usual routes including oral, i.p., or continuous infusion using implantable pumps. Tumor growth is followed over time using calipers. Further, tumor samples can be harvested at any time after the initiation of treatment for analysis as described above (Example B) to evaluate compound effects on JAK activity and downstream signaling pathways. In addition, selectivity of the compound(s) can be assessed using xenograft tumor models that are driven by other know kinases (e.g. Bcr-Abl) such as the K562 tumor model.

### Example D: Murine Skin Contact Delayed Hypersensitivity Response Test

Compounds herein can also be tested for their efficacies (of inhibiting JAK targets) in the T-cell driven murine delayed hypersensitivity test model. The murine skin contact delayed-type hypersensitivity (DTH) response is considered to be a valid model of clinical contact dermatitis, and other T-lymphocyte mediated immune disorders of the skin, such as psoriasis (Immunol Today. 1998 January;19 (1): 37-44). Murine DTH shares multiple characteristics with psoriasis, including the immune infiltrate, the accompanying increase in inflammatory cytokines, and keratinocyte hyperproliferation. Furthermore, many classes of agents that are efficacious in treating psoriasis in the clinic are also effective inhibitors of the DTH response in mice (Agents Actions. 1993 January;38 (1-2): 116-21).

On Day 0 and 1, Balb/c mice are sensitized with a topical application, to their shaved abdomen with the antigen 2,4, dinitro-fluorobenzene (DNFB). On day 5, ears are measured for thickness using an engineer's micrometer. This measurement is recorded and used as a baseline. Both of the animals' ears are then challenged by a topical application of DNFB in a total of 20 μL (10 μL on the internal pinna and 10 μL on the external pinna) at a concentration of 0.2%. Twenty-four to seventy-two hours after the challenge, ears are measured again. Treatment with the test compounds is given throughout the sensitization and challenge phases (day-1 to day 7) or prior to and throughout the challenge phase (usually afternoon of day 4 to day 7). Treatment of the test compounds (in different concentration) is administered either systemically or topically (topical application of the treatment to the ears). Efficacies of the test compounds are indicated by a reduction in ear swelling comparing to the situation without the treatment. Compounds causing a reduction of 20% or more were considered efficacious. In some experiments, the mice are challenged but not sensitized (negative control).

The inhibitive effect (inhibiting activation of the JAK-STAT pathways) of the test compounds can be confirmed by immunohistochemical analysis. Activation of the JAK-STAT pathway(s) results in the formation and translocation of functional transcription factors. Further, the influx of immune cells and the increased proliferation of keratinocytes should also provide unique expression profile changes in the ear that can be investigated and quantified. Formalin fixed and paraffin embedded ear sections (harvested after the challenge phase in the DTH model) are subjected to immunohistochemical analysis using an antibody that specifically interacts with phosphorylated STAT3 (clone 58E12, Cell Signaling Technologies). The mouse ears are treated with test compounds, vehicle, or dexamethasone (a clinically efficacious treatment for psoriasis), or without any treatment, in the DTH model for comparisons. Test compounds and the dexamethasone can produce similar transcriptional changes both qualitatively and quantitatively, and both the test compounds and dexamethasone can reduce the number of infiltrating cells. Both systemically and topical administration of the test compounds can produce inhibitive effects, i.e., reduction in the number of infiltrating cells and inhibition of the transcriptional changes.

### Example E: In Vivo Anti-Inflammatory Activity

Compounds herein can be evaluated in rodent or non-rodent models designed to replicate a single or complex inflammation response. For instance, rodent models of arthritis can be used to evaluate the therapeutic potential of compounds dosed preventatively or therapeutically. These models include but are not limited to mouse or rat collagen-induced arthritis, rat adjuvant-induced arthritis, and collagen antibody-induced arthritis. Autoimmune diseases including, but not limited to, multiple sclerosis, type I-diabetes mellitus, uveoretinitis, thyroditis, myasthenia gravis, immunoglobulin nephropathies, myocarditis, airway sensitization (asthma), lupus, or colitis may also be used to evaluate the therapeutic potential of compounds herein. These models are well established in the research community and are familiar to those schooled in the art (Current Protocols in Immunology, Vol 3., Coligan, J. E. et al, Wiley Press.; Methods in Molecular Biology: Vol. 225, Inflammation Protocols., Winyard, P. G. and Willoughby, D. A., Humana Press, 2003.).

### Example F: Animal Models for the Treatment of Dry Eye, Uveitis, and Conjunctivitis

Agents may be evaluated in one or more preclinical models of dry eye known to those schooled in the art including, but not limited to, the rabbit concanavalin A (ConA) lacrimal gland model, the scopolamine mouse model (subcutaneous or transdermal), the Botulinumn mouse lacrimal gland model, or any of a number of spontaneous rodent auto-immune models that result in ocular gland dysfunction (e.g. NOD-SCID, MRL/lpr, or NZB/NZW) (Barabino et al., Experimental Eye Research 2004, 79, 613-621 and Schrader et al., Developmental Opthalmology, Karger 2008, 41, 298-312, each of which is incorporated herein by reference in its entirety). Endpoints in these models may include histopathology of the ocular glands and eye (cornea, etc.) and possibly the classic Schirmer test or modified versions thereof (Barabino et al.) which measure tear production. Activity may be assessed by dosing via multiple routes of administration (e.g. systemic or topical) which may begin prior to or after measurable disease exists.

Agents may be evaluated in one or more preclinical models of uveitis known to those schooled in the art. These include, but are not limited to, models of experimental autoimmune uveitis (EAU) and endotoxin induced uveitis (EIU). EAU experiements may be performed in the rabbit, rat, or mouse and may involve passive or activate immunization. For instance, any of a number or retinal antigens may be used to sensitize animals to a relevant immunogen after which animals may be challenged ocuarly with the same antigen. The EIU model is more acute and involves local or systemic administration of lipopolysaccaride at sublethal doses. Endpoints for both the EIU and EAU models may include fundoscopic exam, histopathology amongst others. These models are reviewed by Smith et al. (Immunology and Cell Biology 1998, 76, 497-512, which is incorporated herein by reference in its entirety). Activity is assessed by dosing via multiple routes of administration (e.g. systemic or topical) which may begin prior to or after measurable disease exists. Some models listed above may also develop scleritis/episcleritis, chorioditis, cyclitis, or iritis and are therefore useful in investigating the potential activity of compounds for the therapeutic treatment of these diseases.

Agents may also be evaluated in one or more preclinical models of conjunctivitis known those schooled in the art. These include, but are not limited to, rodent models utilizing guinea-pig, rat, or mouse. The guinea-pig models include those utilizing active or passive immunization and/or immune challenge protocols with antigens such as ovalbumin or ragweed (reviewed in Groneberg, D. A., et al., Allergy 2003, 58, 1101-1113, which is incorporated herein by reference in its entirety). Rat and mouse models are similar in general design to those in the guinea-pig (also reviewed by Groneberg). Activity may be assessed by dosing via multiple routes of administration (e.g. systemic or topical) which may begin prior to or after measurable disease exists. Endpoints for such studies may include, for example, histological, immunological, biochemical, or molecular analysis of ocular tissues such as the conjunctiva.

### Example G: In Vivo Protection of Bone

Compounds may be evaluated in various preclinical models of osteopenia, osteoporosis, or bone resorption known to those schooled in the art. For example, ovariectomized rodents may be used to evaluate the ability of compounds to affect signs and markers of bone remodeling and/or density (W. S. S. Jee and W. Yao, J Musculoskel. Nueron. Interact., 2001, 1(3), 193-207, which is incorporated herein by reference in its entirety). Alternatively, bone density and architecture may be evaluated in control or compound treated rodents in models of therapy (e.g. glucocorticoid) induced osteopenia (Yao, et al. Arthritis and Rheumatism, 2008, 58(6), 3485-3497; and id. 58(11), 1674-1686,both of which are incorporated herein by reference in its entirety). In addition, the effects of compounds on bone resorption and density may be evaluable in the rodent models of arthritis discussed above (Example E). Endpoints for all these models may vary but often include histological and radiological assessments as well as immunohisotology and appropriate biochemical markers of bone remodeling.

1.-45. (canceled)

46. One or more sustained release tablets, suitable for oral administration, comprising:
{1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof; and
a means for providing a ratio of mean peak plasma concentration (Cₘₐₓ) to mean 12-hour plasma concentration (C₁₂ₕ) of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile of 9 to 40 to a fasted individual.

47. The one or more sustained release tablets of claim 46, comprising a means for providing a ratio of mean peak plasma concentration (Cₘₐₓ) to mean 12-hour plasma concentration (C₁₂ₕ) of {1-{1-[3-fluoro-2-(trifluoromethyl)isonicotinoyl]piperidin-4-yl}-3-[4-(7H-pyrrolo[2,3-d]pyrimidin-4-yl)-1H- pyrazol-1-yl]azetidin-3-yl}acetonitrile of 15 to 30 to a fasted individual.

48. The one or more sustained release tablets of claim 46, comprising a means for providing a ratio of mean peak plasma concentration (Cₘₐₓ) to mean 12-hour plasma concentration (C₁₂ₕ) of {1-{1-[3-fluoro-2-(trifluoromethyl)isonicotinoyl]piperidin-4-yl}-3-[4-(7H-pyrrolo[2,3-d]pyrimidin-4-yl)-1H- pyrazol-1-yl]azetidin-3-yl}acetonitrile of 10 to 70 to an individual after a high-fat meal.

49. The one or more sustained release tablets of claim 46, comprising a means for providing a ratio of mean peak plasma concentration (Cₘₐₓ) to mean 12-hour plasma concentration (C₁₂ₕ) of {1-{1-[3-fluoro-2-(trifluoromethyl)isonicotinoyl]piperidin-4-yl}-3-[4-(7H-pyrrolo[2,3-d]pyrimidin-4-yl)-1H- pyrazol-1-yl]azetidin-3-yl}acetonitrile of 15 to 50 to an individual after a high-fat meal.

50. The one or more sustained release tablets of claim 46, comprising a means for providing a ratio of mean peak plasma concentration (Cₘₐₓ) to mean 12-hour plasma concentration (C₁₂ₕ) of {1-{1-[3-fluoro-2-(trifluoromethyl)isonicotinoyl]piperidin-4-yl}-3-[4-(7H-pyrrolo[2,3-d]pyrimidin-4-yl)-1H- pyrazol-1-yl]azetidin-3-yl}acetonitrile of 25 to 45 to an individual after a high-fat meal.

51. One or more sustained release tablets, suitable for oral administration, comprising:
{1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof; and
means for providing:
(a) a mean peak plasma concentration (Cₘₐₓ) of {1-{1-[3-fluoro-2-(trifluoromethyl)isonicotinoyl]piperidin-4-yl}-3-[4-(7H-pyrrolo[2,3-d]pyrimidin- 4-yl)-1H-pyrazol-1-yl]azetidin-3-yl}acetonitrile of 0.191 μM±0.10 μM to a fasted individual; or
(b) a mean time to peak plasma concentration (Tₘₐₓ) of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile of 0.5 hours to 3 hours to a fasted individual; or
(c) a mean half-life (t_{1/2}) of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile of 1 hour to 20 hours to a fasted individual; or
(d) a mean half-life (t_{1/2}) of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile of 1 hour to 7 hours to an individual after a high-fat meal; or
(e) any combination of (a)-(d).

52. The one or more sustained release tablets of claim 51, comprising a means for providing a mean half-life (t_{1/2}) of {1-{1-[3-fluoro-2-(trifluoromethyl)isonicotinoyl]piperidin-4-y1}-3-[4-(7H-pyrrolo[2,3-d]pyrimidin-4- yl)-1H-pyrazol-1-yl]azetidin-3-yl}acetonitrile of 2 hour to 5 hours to an individual after a high-fat meal.

53. The one or more sustained release tablets of claim 51, comprising a means for providing a mean half-life (t_{1/2}) of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile of 4.9 h±2.6 h to a fasted individual.

54. The one or more sustained release tablets of claim 51, comprising a means for providing a mean peak plasma concentration (Cₘₐₓ) of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile of 0.191 μM±0.10 μM to a fasted individual.

55. The one or more sustained release tablets of claim 51, comprising a means for providing a mean time to peak plasma concentration (Tₘₐₓ) of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile of 0.5 hours to 3 hours to a fasted individual.

56. The one or more sustained release tablets of claim 51, comprising a means for providing a mean half-life (t_{1/2}) of {1-{1-[3-fluoro-2-(trifluoromethyl)isonicotinoyl]piperidin-4-y1}-3-[4-(7H-pyrrolo[2,3-d]pyrimidin-4- yl)-1H-pyrazol-1-yl]azetidin-3-yl}acetonitrile of 1 hour to 20 hours to a fasted individual.

57. The one or more sustained release tablets of claim 51, comprising a means for providing a mean half-life (t_{1/2}) of {1-{1-[3-fluoro-2-(trifluoromethyl)isonicotinoyl]piperidin-4-y1}-3-[4-(7H-pyrrolo[2,3-d]pyrimidin-4- yl)-1H-pyrazol-1-yl]azetidin-3-yl}acetonitrile of 1 hour to 7 hours to an individual after a high-fat meal.

58. The one or more sustained release tablets of claim 46, wherein the salt is {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile adipic acid salt.

59. The one or more sustained release tablets of claim 51, wherein the salt is {1-{1-[3-fluoro-2-(trifluoromethyl)isonicotinoyl]piperidin-4-yl}-3-[4-(7H-pyrrolo[2,3-d]pyrimidin-4-y1)-1H-pyrazol-1-yl]azetidin-3-yl}acetonitrile adipic acid salt.
